SEQUENCE LISTING (91 sequences)

SEQ ID NO: 1 - RNA, 1776 nt, Hepatitis C virus
ucgauguccuacacauggacaggcgcccugaucacgccaugcgcugcggaggaauccaagcugcccaucaaugcacugagcaacucuuugcuccgucaccacaacauggucuaugcuacaacaucucgcagcgcaagccugcggcagaagaaggucaccuuugacagacugcagguccuggacgaccacuaccgggacgugcucaaggagaugaaggcgaaggcguccacaguuaaggcuaaacuucuauccguggaggaagccuguaagcugacgcccccacauucggccaaaucuaaauuuggcuauggggcaaaggacguccggaaccuauccagcaaggccguuaaccacauccgcuccguguggaaggacuugcuggaagacacugagacaccaauugacaccaccaucauggcaaaaaaugagguuuucugcguccaaccagagaaggggggccgcaagccagcucgccuuaucguauucccagauuuggggguucgugugugcgagaaaauggcccuuuacgauguggucuccacccucccucaggccgugaugggcucuucauacggauuccaguacucuccuggacagcgggucgaguuccuggugaauaccuggaaaucgaaaaaaugcccuaugggcuucucauaugacacccgcugcuuugacucaacggucacugagaaugacauccguguugaggagucaaucuaccaauguugugacuuggcccccgaagccagacaggccauaaggucgcucacagagcggcuuuacaucgggggcccccugacuaauucaaaggggcagaacugcggcuaucgccggugccgcgcgagcgguguacugacgaccagcugcgguaauacccucacauguuacuugaaggccucugcggccugucgagcugcgaagcuccaggacugcacgaugcucgugugcggagacgaccuugucguuaucugugaaagcgcggggacccaagaggacgcggcgagccuacgagccuucacggaggcuaugacuagauacucugcccccccuggggacccgccccaaccagaauacgacuuggaguugauaacaucaugcuccuccaaugugucugucgcgcacgaugcaucuggcaaaaggguguacuaucucacccgugaccccaccaccccccuugcgcgggcugcgugggagacagcuagacacacuccagucaauuccuggcuaggcaacaucaucauguaugcgcccaccuugugggcaaggaugauccugaugacucauuucuucuccauccuucuagcucaggaacaacuugaaaaagcccuagauugucagaucuacggggccuguuacuccauugagccacuugaccuaccucagaucauucaacgacuccauggccuuagcgcauuuucacuccauaguuacucuccaggugagaucaauaggguggcuucaugccucaggaaacuugggguaccgcccuugcgagucuggagacaucgggccagaaguguccgcgcuaggcuacugucccagggggggagggcugccacuuguggcaaguaccucuucaacugggcaguaaggaccaagcucaaacucacuccaaucccggcugcgucccaguuggauuuauccagcugguucguugcugguuacagcgggggagacauauaucacagccugucucgugcccgaccccgcugguucauguggugccuacuccuacuuucuguagggguaggcaucuaccugcuccccaaccgguga

SEQ ID NO: 2 - RNA, 23 nt, Hepatitis C virus
aagccagcucgccuuaucguauu

SEQ ID NO: 3 - RNA, 23 nt, Hepatitis C virus
aaggucgcucacagagcggcuuu

SEQ ID NO: 4 - RNA, 23 nt, Hepatitis C virus
aaggaugauccugaugacucauu

SEQ ID NO: 5 - RNA, 23 nt, Hepatitis C virus
aaggguguacuaucucacccgug

SEQ ID NO: 6 - RNA, 23 nt, Hepatitis C virus
aaauggcccuuuacgaugugguc

SEQ ID NO: 7 - RNA, 23 nt, Hepatitis C virus
aaugacauccguguugaggaguc

SEQ ID NO: 8 - RNA, 23 nt, Hepatitis C virus
aaggaccaagcucaaacucacuc

SEQ ID NO: 9 - RNA, 23 nt, Hepatitis C virus
aaccagaauacgacuuggaguug

SEQ ID NO: 10 - RNA, 23 nt, Hepatitis C virus
aaggagaugaaggcgaaggcguc

SEQ ID NO: 11 - RNA, 23 nt, Hepatitis C virus
aaggccguuaaccacauccgcuc

SEQ ID NO: 12 - RNA, 23 nt, Hepatitis C virus
aaaaaaugcccuaugggcuucuc

SEQ ID NO: 13 - RNA, 23 nt, Hepatitis C virus
aaaggggcagaacugcggcuauc

SEQ ID NO: 14 - RNA, 23 nt, Hepatitis C virus
aaaugcccuaugggcuucucaua

SEQ ID NO: 15 - RNA, 23 nt, Hepatitis C virus
aacauggucuaugcuacaacauc

SEQ ID NO: 16 - RNA, 23 nt, Hepatitis C virus
aaaaagcccuagauugucagauc

SEQ ID NO: 17 - RNA, 23 nt, Hepatitis C virus
aaagcccuagauugucagaucua

SEQ ID NO: 18 - RNA, 23 nt, Hepatitis C virus
aauaccuggaaaucgaaaaaaug

SEQ ID NO: 19 - RNA, 23 nt, Hepatitis C virus
cauguggugccuacuccuacuuu

SEQ ID NO: 20 - RNA, 23 nt, Hepatitis C virus
gacuccauggccuuagcgcauuu

SEQ ID NO: 21 - RNA, 23 nt, Hepatitis C virus
caucaaugcacugagcaacucuu

SEQ ID NO: 22 - RNA, 23 nt, Hepatitis C virus
caaccagaauacgacuuggaguu

SEQ ID NO: 23 - RNA, 23 nt, Hepatitis C virus
cacuugaccuaccucagaucauu

SEQ ID NO: 24 - RNA, 23 nt, Hepatitis C virus
caguuggauuuauccagcugguu

SEQ ID NO: 25 - RNA, 23 nt, Hepatitis C virus
uaaggucgcucacagagcggcuu

SEQ ID NO: 26 - RNA, 23 nt, Hepatitis C virus
caucgggggcccccugacuaauu

SEQ ID NO: 27 - RNA, 23 nt, Hepatitis C virus
uagcgcauuuucacuccauaguu

SEQ ID NO: 28 - RNA, 23 nt, Hepatitis C virus
cacauucggccaaaucuaaauuu

SEQ ID NO: 29 - RNA, 23 nt, Hepatitis C virus
caucauggcaaaaaaugagguuu

SEQ ID NO: 30 - RNA, 23 nt, Hepatitis C virus
gacgcggcgagccuacgagccuu

SEQ ID NO: 31 - RNA, 23 nt, Hepatitis C virus
caccaauugacaccaccaucaug

SEQ ID NO: 32 - RNA, 23 nt, Hepatitis C virus
cacugagaaugacauccguguug

SEQ ID NO: 33 - RNA, 23 nt, Hepatitis C virus
cacauguuacuugaaggccucug

SEQ ID NO: 34 - RNA, 23 nt, Hepatitis C virus
cauauaucacagccugucucgug

SEQ ID NO: 35 - RNA, 23 nt, Hepatitis C virus
caacggucacugagaaugacauc

SEQ ID NO: 36 - RNA, 23 nt, Hepatitis C virus
caugcuccuccaaugugucuguc

SEQ ID NO: 37 - RNA, 23 nt, Hepatitis C virus
cauugagccacuugaccuaccuc

SEQ ID NO: 38 - RNA, 23 nt, Hepatitis C virus
caguaaggaccaagcucaaacuc

SEQ ID NO: 39 - RNA, 23 nt, Hepatitis C virus
cauucaacgacuccauggccuua

SEQ ID NO: 40 - RNA, 23 nt, Hepatitis C virus
caugccucaggaaacuuggggua

SEQ ID NO: 41 - RNA, 23 nt, Hepatitis C virus
gaaggacuugcuggaagacacug

SEQ ID NO: 42 - RNA, 23 nt, Hepatitis C virus
gagaucaauaggguggcuucaug

SEQ ID NO: 43 - RNA, 23 nt, Hepatitis C virus
gaaggcguccacaguuaaggcua

SEQ ID NO: 44 - RNA, 23 nt, Hepatitis C virus
gaaccuauccagcaaggccguua

SEQ ID NO: 45 - RNA, 23 nt, Hepatitis C virus
gaugcaucuggcaaaagggugua

SEQ ID NO: 46 - RNA, 23 nt, Hepatitis C virus
uaugacacccgcugcuuugacuc

SEQ ID NO: 47 - RNA, 23 nt, Hepatitis C virus
uacuuucuguagggguaggcauc

SEQ ID NO: 48 - RNA, 23 nt, Hepatitis C virus
caccaccaucauggcaaaaaaug

SEQ ID NO: 49 - RNA, 23 nt, Hepatitis C virus
caaugugucugucgcgcacgaug

SEQ ID NO: 50 - RNA, 23 nt, Hepatitis C virus
caaacucacuccaaucccggcug

SEQ ID NO: 51 - RNA, 23 nt, Hepatitis C virus
cauggucuaugcuacaacaucuc

SEQ ID NO: 52 - RNA, 23 nt, Hepatitis C virus
cagaucuacggggccuguuacuc

SEQ ID NO: 53 - RNA, 23 nt, Hepatitis C virus
caggccgugaugggcucuucaua

SEQ ID NO: 54 - RNA, 23 nt, Hepatitis C virus
gaggagucaaucuaccaauguug

SEQ ID NO: 55 - RNA, 23 nt, Hepatitis C virus
gauaacaucaugcuccuccaaug

SEQ ID NO: 56 - RNA, 23 nt, Hepatitis C virus
gauugucagaucuacggggccug

SEQ ID NO: 57 - RNA, 23 nt, Hepatitis C virus
gaagccagacaggccauaagguc

SEQ ID NO: 58 - RNA, 23 nt, Hepatitis C virus
gaguugauaacaucaugcuccuc

SEQ ID NO: 59 - RNA, 23 nt, Hepatitis C virus
gacucauuucuucuccauccuuc

SEQ ID NO: 60 - RNA, 23 nt, Hepatitis C virus
gaaggcgaaggcguccacaguua

SEQ ID NO: 61 - RNA, 23 nt, Hepatitis C virus
uacgagccuucacggaggcuaug

SEQ ID NO: 62 - RNA, 23 nt, Hepatitis C virus
uaacaucaugcuccuccaaugug

SEQ ID NO: 63 - RNA, 23 nt, Hepatitis C virus
uaccucagaucauucaacgacuc

SEQ ID NO: 64 - RNA, 23 nt, Hepatitis C virus
uagggguaggcaucuaccugcuc

SEQ ID NO: 65 - RNA, 23 nt, Hepatitis C virus
uacuccuacuuucuguaggggua

SEQ ID NO: 66 - RNA, 23 nt, Hepatitis C virus
caacaucucgcagcgcaagccug

SEQ ID NO: 67 - RNA, 23 nt, Hepatitis C virus
caaucuaccaauguugugacuug

SEQ ID NO: 68 - RNA, 23 nt, Hepatitis C virus
caggacugcacgaugcucgugug

SEQ ID NO: 69 - RNA, 23 nt, Hepatitis C virus
caacuugaaaaagcccuagauug

SEQ ID NO: 70 - RNA, 23 nt, Hepatitis C virus
caagccugcggcagaagaagguc

SEQ ID NO: 71 - RNA, 23 nt, Hepatitis C virus
caaaaaaugagguuuucugcguc

SEQ ID NO: 72 - RNA, 23 nt, Hepatitis C virus
cagaauacgacuuggaguugaua

SEQ ID NO: 73 - RNA, 23 nt, Hepatitis C virus
gacuagauacucugcccccccug

SEQ ID NO: 74 - RNA, 23 nt, Hepatitis C virus
gacuuggaguugauaacaucaug

SEQ ID NO: 75 - RNA, 23 nt, Hepatitis C virus
gaaguguccgcgcuaggcuacug

SEQ ID NO: 76 - RNA, 23 nt, Hepatitis C virus
uacuugaaggccucugcggccug

SEQ ID NO: 77 - RNA, 23 nt, Hepatitis C virus
uacgacuuggaguugauaacauc

SEQ ID NO: 78 - RNA, 23 nt, Hepatitis C virus
cagaacugcggcuaucgccggug

SEQ ID NO: 79 - RNA, 23 nt, Hepatitis C virus
caaucccggcugcgucccaguug

SEQ ID NO: 80 - RNA, 23 nt, Hepatitis C virus
gacagcgggucgaguuccuggug

SEQ ID NO: 81 - RNA, 23 nt, Hepatitis C virus
cagggggggagggcugccacuug

SEQ ID NO: 82 - RNA, 23 nt, Hepatitis C virus
gaaggggggccgcaagccagcuc

SEQ ID NO: 83 - RNA, 23 nt, Hepatitis C virus
caccaccccccuugcgcgggcug

SEQ ID NO: 84 - DNA, 21 bp, Artificial Sequence
  Description of Combined DNA/RNA Molecule synthetic DNA/RNA hybrid
cuggacuuccagaagaacatt

SEQ ID NO: 85 - DNA, 21 bp, Artificial Sequence
  Description of Combined DNA/RNA Molecule synthetic DNA/RNA hybrid
uguucuucuggaaguccagtt

SEQ ID NO: 86 - DNA, 23 bp, Artificial Sequence
  Description of Combined DNA/RNA Molecule synthetic DNA/RNA hybrid
cuggacuuccagaagaacatttt

SEQ ID NO: 87 - DNA, 23 bp, Artificial Sequence
  Description of Combined DNA/RNA Molecule synthetic DNA/RNA hybrid
uguucuucuggaaguccagtttt

SEQ ID NO: 88 - DNA, 21 bp, Artificial Sequence
  Description of Combined DNA/RNA Molecule synthetic DNA/RNA hybrid
cuggacuuccagaagaacatt

SEQ ID NO: 89 - DNA, 21 bp, Artificial Sequence
  Description of Combined DNA/RNA Molecule synthetic DNA/RNA hybrid
uguucuucuggaaguccagtt

SEQ ID NO: 90 - DNA, 18 bp, Artificial Sequence
  Description of Combined DNA/RNA Molecule synthetic DNA/RNA hybrid
gacuuccagaagaacauu

SEQ ID NO: 91 - DNA, 21 bp, Artificial Sequence
  Description of Combined DNA/RNA Molecule synthetic DNA/RNA hybrid
uguucuucuggaaguccaguu

(19) United States
(12) Patent Application Publication
(10) Pub. No.: US 2008/0188429 A1
(43) Pub. Date: Aug. 7, 2008

(54) Synthetic siRNA compounds and methods for the downregulation of gene expression

(76) Inventor: Radhakrishnan P. Iyer, Shrewsbury, MA (US)

Correspondence Address: ELMORE PATENT LAW GROUP, PC, 515 Groton Road, Unit 1R, Westford, MA, 01886, US

(21) Appl. No.: 10/746,951

(22) Filed: Dec. 23, 2003

Related U.S. Application Data
(60) Provisional application No. 60/436,599, filed on Dec. 27, 2002.

(51) Int. Cl. A61K 31/7088; C07H 21/02; C12N 5/02

(52) U.S. Cl. 514/44; 536/24.5; 435/375

(57) ABSTRACT

This invention relates to the design and synthesis of chemically modified short interfering nucleic acid (siNA) compounds capable of mediating RNA interference (RNAi) against target genes.

### RELATED APPLICATION

This application claims priority to U.S. Ser. No. 60/436,599, filed on Dec. 27, 2002. The entire teachings of the above application are incorporated herein by reference.

### FIELD OF THE INVENTION

This invention relates to the design and synthesis of chemically modified short interfering nucleic acid (siNA) compounds capable of mediating RNA interference (RNAi) against target genes.

### BACKGROUND OF THE INVENTION

Regulation of gene expression by “silencing” of RNA was first discovered in plants in the 1990s (1-4). Thus, in early experiments with purple colored petunias, transgenic plants were created, that carried an extra copy of the “color” gene that would enable the production of deeper coloring flowers. However, the resulting plants had white flowers instead of the anticipated deeply purple-colored flowers. This observation was interpreted as being the result of post-transcriptional silencing of the transgene, as well as, the cellular gene that encode the gene product. The suppression of gene expression was further traced to sequence-specific degradation of the corresponding mRNA without alteration in the rate of transcription of the gene. Thus, even though the gene was transcribed and processed into mRNA, the mRNA was destroyed in the cytoplasm as quickly as it was made. This phenomenon is referred to as RNA interference (RNAi) and has been shown to involve the intermediacy of double-stranded RNA (dsRNA) (1). In summary, RNA interference is a post-transcriptional gene-silencing (PTGS) event in which both the transgene and the homologous chromosomal loci appear to be co-suppressed by the corresponding dsRNA. RNAi appears to be present in almost all eukaryotic systems. It is believed that the phenomenon of RNAi is utilized by nature to protect its genome from attack by mobile genetic elements such as viruses and transposons. In nature, repetitive and mobile genetic elements such as viruses and transposons can integrate near the promoters of cellular genes and be transcribed to dsRNA to produce RNAi effect (1). Importantly, cytoplasmically replicating RNA viruses can act as both targets and inducers of PTGS.

Understanding the mechanism by which dsRNA triggers RNA silencing has been crucial to unraveling the potential therapeutic and diagnostic application of RNAi. Using an in vitro system involving D. melanogaster, Tuschl et al., (6) have discovered that before RNAi occurs, dsRNA is first cleaved by specific nucleases at regular intervals to generate 21 to 23 nucleotide pieces (NT) (Scheme 1). It is believed that these short oligoribonucleotides derived from dsRNA are the likely intermediates of RNA interference and are termed short interfering RNAs (siRNA). The nuclease is believed to be an ATP-dependent ribonuclease called “Dicer” that belongs to an RNase III family of double-stranded RNA-specific endonuclease. Recently, an RNAse III protein has also been characterized that contains helicase, RNAse III, and dsRNA motifs (1).

Further evidence for the intermediacy of siRNA is based on several observations: (a) precursor dsRNA of less than 38 base pairs in length are inefficient mediators of RNAi because the rate of siRNA formation appears to be significantly reduced in comparison to longer dsRNA; (b) in experiments using D. melanogaster embryos it was found that the siRNA generated from dsRNA usually carry a 5′-monophosphate and a free 3′-hydroxyl group and that processing of dsRNA occurs with no apparent sequence preference (8); (c) The siRNA that are 21 to 23 double stranded ribonucleotides, carry two nucleotide overhangs at each of the 3′-ends and RNAse III enzyme is known to cleave dsRNA to generate fragments with such 2 to 3 NT overhangs (8); (d) The siRNA was detected in vivo in D. melanogaster embryos and C. elegans adults when dsRNA was injected; (e) Interestingly it was observed that chemically synthesized siRNAs were also capable of effecting target RNA cleavage in vitro; and (f) It has been demonstrated that siRNAs induce sequence-specific RNAi in mammalian systems as well (7). Taken together, these observations suggest that an RNase III-like enzyme is responsible for processing of dsRNA into siRNA and that siRNA is the intermediate for RNAi.

FIG. 1 shows the mechanism of RNA interference. Double stranded RNA is processed to siRNA by Dicer and siRNA as RISC complex cleaves the target RNA. Once formed, these 21 to 23 NT siRNA could serve as guide sequences in complexing with mRNA and target it for degradation. siRNA complexes with proteins, to form mRNA-cleaving RNA-protein complexes, alternatively known as RNA-induced silencing complex (RISC) that have endoribonuclease activity different from Dicer. Using siRNA as a guide, the RISC then cleaves the corresponding mRNA at the site that is complementary to the siRNA. The position of mRNA cleavage is within the binding segment of mRNA that is complexed with the antisense strand of siRNA and therefore suggests a hybridization-triggered induction of cleavage. Biochemical analysis shows that both sense and antisense strands within siRNA have distinct roles for the functional activity of RISC. Presumably, the antisense strand complexes with target mRNA and induces its cleavage whereas the sense strand participates in siRNA duplex formation. It is the duplex structure that is recognized by RISC proteins which has RNase III, helicase and ATPase activity (1).

There appears to be specific advantages in using siRNA for RNAi effect. Thus, in early experiments with mammalian cells that used dsRNA, it was found that dsRNA induces dsRNA-dependent protein kinase (PKR), which phosphorylates and inactivates the translation factor elF2a leading to a general non-specific suppression of protein synthesis and apoptosis. In contrast, Tuschl et al., (6) have shown that 21 to 23 NT duplexes can function as siRNA and induce sequence-specific and selective RNAi in mammalian cell lines such as human kidney cells and HeLa cells (7). Thus, the use of siRNA appears to overcome the problems in the use of dsRNA and may become the method of choice for therapeutics, diagnostics, and functional genomics via RNAi mechanism.

The key is to discover the siRNA, which has structural attributes and pharmaceutical properties (enzymatic stability, deliverability and favorable pharmacokinetic and pharmacodynamic parameters) that combine RNAi-functional competency, potency and selectivity.

### SUMMARY OF THE INVENTION

The present invention provides chemically modified small interfering RNA (siRNA) compositions that possess particularly desirable structural attributes and pharmaceutical properties (e.g. enzymatic stability, deliverability and favorable pharmacokinetic and pharmacodynamic parameters). The siRNA compositions of the invention are capable of mediating selective and specific down regulation of a target gene via the RNA interference mechanism or by mechanisms related to RNAi. The compositions and methods of the invention are particularly useful as pharmaceuticals and therapeutics.

### BRIEF DESCRIPTION OF THE DRAWINGS

FIG. 1 is a diagram showing the mechanism of RNA interference.

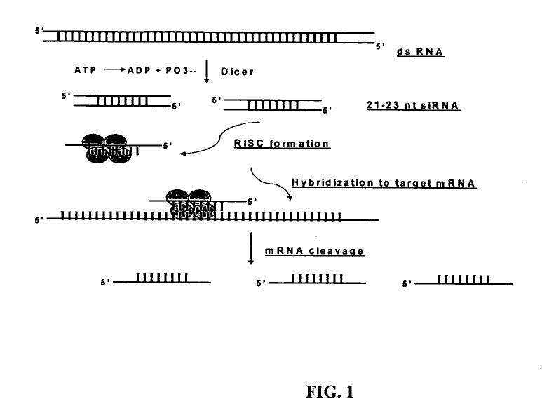

FIG. 2 is a diagram showing non-limiting examples of hairpin siRNA of the invention.

FIG. 3 is a diagram showing non-limiting examples of linear siRNA of the invention.

FIG. 4 is a diagram showing non-limiting examples of linear siRNA of the invention.

FIG. 5 is a diagram showing one embodiment of the SiRNA of the invention.

### DETAILED DESCRIPTION OF THE INVENTION

The present invention provides chemically modified siRNA molecules capable of mediating RNA interference (RNAi) against a target gene. In one embodiment, the invention provides a chemically modified short interfering nucleic acid molecule capable of down regulating the expression of a target gene by RNAi, wherein the molecule comprises a non-nucleotidic trivalent linker having three terminal ends, wherein a first oligonucleotide is attached to a first terminal end and a second oligonucleotide is attached to a second terminal end and a hydrophobic moiety is attached to a third terminal end and wherein at least one of the first or second oligonucleotides, is complementary to or homologous with, the target gene. In one embodiment the molecule has the structure of Formula 1:

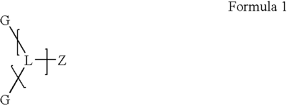

wherein
G are each independently an oligonucleotide;
L is a trivalent spacer; and
Z is a hydrophobic moiety,
wherein at least one of G is complementary to, or homologous with, a target gene.

In one embodiment, the hydrophobic moiety is optionally attached to a terminal end via a spacer moiety. The spacer moiety may be a short or long chain spacer moiety.

Preferred long chain spacer moieties include aliphatic or substituted aliphatic spacers optionally interrupted by one or more heteroatoms. In another embodiment, the long chain spacer moiety may be an aralkyl moiety or substituted aralkyl moiety optionally interrupted by one or more heteroatoms. The trivalent linker may preferably be a glycol moiety, a phosphate moiety, a sulfonamide moiety, a carbamate moiety or an alkanol amine moiety.

In one preferred embodiment, the molecule may have the structure of Formula 3:

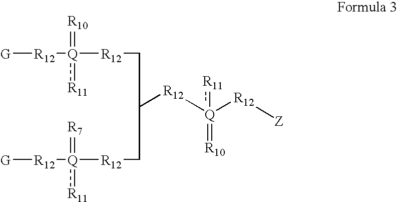

wherein each Q is independently a hexavalent atom such as sulfur, a pentavalent atom such as phosphorus, or a tetravalent atom such as sulfur, or carbon in which case the bond between Q and R₁₁ does not exist;

each R₁₀ is optionally O, S or NH except that when Q=S, R₁₀═O, R₁₁═O and R₁₂ is optionally O or NH;

each R₁₂ is optionally O, S, or NH;
each R₁₁ is O;
G is an oligonucleotide; and
Z is a hydrophobic moiety.

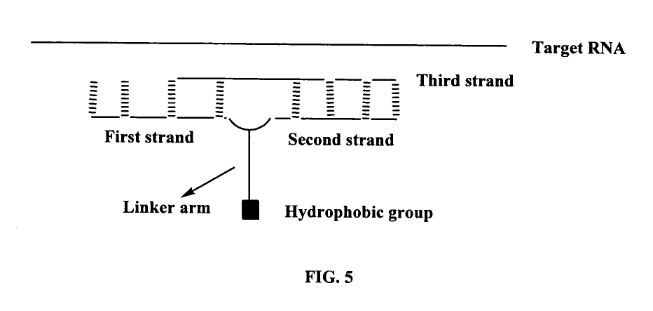

In another embodiment, the siRNA molecule of the invention comprises a third oligonucleotide. In this embodiment, each of the first and second oligonucleotides attached to the trivalent linker is complementary to a portion of the third oligonucleotide and, when taken together with the trivalent linker to which they are attached, each of the first and second oligonucleotides form a double stranded siRNA molecule with the third oligonucleotide. Preferably either or both of the first and second oligonucleotides when taken together with the trivalent linker to which they are attached are complementary to, or homologous with, a target gene. One non-limiting example of a siRNA molecule of the invention having these features is shown in FIG. 5. For ease of reference, siRNA molecules of the invention comprising first and second oligonucleotides taken together with their trivalent linker that are hybridized to a third oligonucleotide are referred to herein as “linear” siRNA molecules of the invention.

In another embodiment, the chemically modified siRNA molecules of the invention comprise a secondary structure. The secondary structure may comprise a single-stranded hairpin structure having a loop region and having self-complementary sense and antisense oligonucleotide regions. In one embodiment, the molecule has the structure of Formula 2:

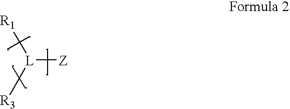

wherein R₁ is an antisense or sense oligonucleotide complementary to R₃, wherein when R₁ is an antisense oligonucleotide, R₁ is also complementary to the target gene;
R₃ is a sense or antisense oligonucleotide complementary to R₁, wherein when R₃ is an antisense oligonucleotide, R₃ is also complementary to a target gene;
L is a trivalent spacer; and
Z is a hydrophobic moiety.

In one embodiment, the hydrophobic moiety is optionally attached to a terminal end via a spacer moiety. The spacer moiety may be a short or long chain spacer moiety. Preferred long chain spacer moieties include aliphatic and substituted aliphatic spacers optionally interrupted by one or more heteroatoms. In another embodiment, the long chain spacer moiety may be an aralkyl moiety or substituted aralkyl moiety optionally interrupted by one or more heteroatoms. The trivalent linker may preferably be a glycol moiety, a phosphate moiety, a sulfonamide moiety, a carbamate moiety or an alkanol amine moiety.

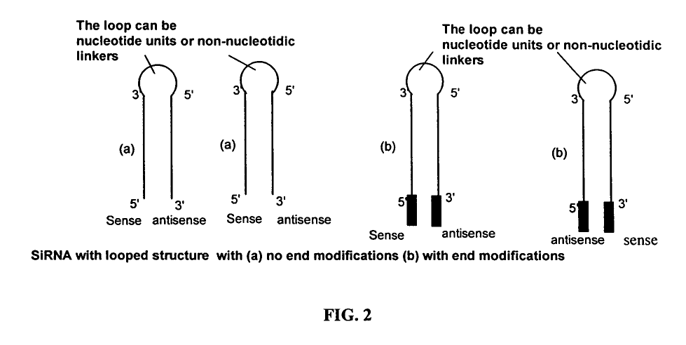

In one embodiment, the loop region of a hairpin siRNA molecule of the invention may comprise nucleotide or non-nucleotide units. Non limiting examples of such structures are shown in FIG. 2. The loop can optionally be made hydrophilic or hydrophobic by incorporating the appropriate chemical moieties as is described in the examples. For example, the loop can be made hydrophilic by incorporating nucleotidic units such as thymidine, with a minimum of 5 to maximum of 8 units that would give a stable loop and also stabilize the duplex structure. The loop can also be made hydrophobic by using cholesterol, polyethylene glycol, and propane diol units. Indeed, the incorporation of some non-nucleotide units and nucleotide units are known to stabilize short hairpin RNA and DNA structures (18). The 3′ and 5′-ends of the stem can also be modified additionally by incorporating capped structures (shown as dark appendages in FIG. 2) and can further protect the strands against nuclease-mediated degradation (3). Non-limiting examples of these hairpin siRNA structures are shown in FIG. 2 and their synthesis is described in the examples.

In one preferred embodiment a hairpin siRNA molecule of the invention has the structure of Formula 4:

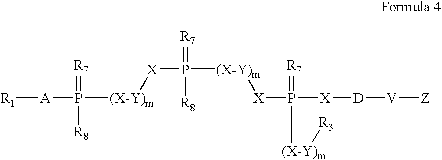

wherein R₁ is a sense or antisense oligonucleotide that is complementary to R₃ and when R₁ is an antisense oligonucleotide, R₁ is also complementary to a target gene;
R₃ is a sense or antisense oligonucleotide that is complementary to R₁ and when R₃ is an antisense oligonucleotide, R₃ is also complementary to a target gene;

### A is S or O

R₇ are each independently S or O;
R₈ are each independently OH, SH, or NR₄R₅;
R₄, R₅, and R₆ are each independently H, alkyl, substituted alkyl, alkaryl or substituted alkaryl, aralkyl or substituted aralkyl;
X are each independently S, O or NR6;

### Y is (CH₂)n;

m is 0-20;
n is 1-20;
n′ is 0 or 1
D is a spacer moiety;
V is an ester moiety or an amide moiety; and
Z is a hydrophobic moiety.

### Preferred

Preferably, spacer moieties include long chain aliphatic or substituted aliphatic spacers optionally interrupted by one or more heteroatoms or an aralkyl moiety or substituted aralkyl moiety optionally interrupted by one or more heteroatoms.

In another embodiment, the molecule has the structure of Formula 5:

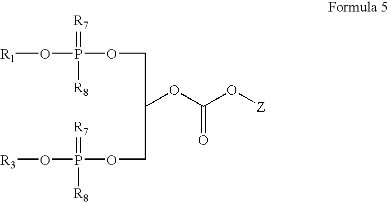

wherein R₁, R₃, R₇, R₈ and Z are as previously defined.

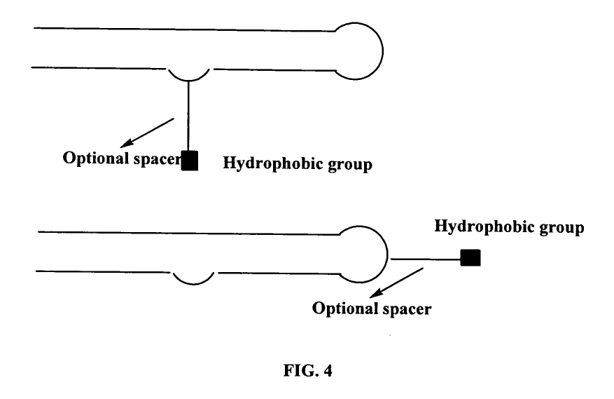

The trivalent linker moiety of Formulas 1, 2, 3, 4, or 5, may appear either in the stem region or the loop region of the siRNA molecule of the invention as is shown in the non-limiting examples of FIG. 4.

Preferred hairpin siRNA molecules of the invention include the molecules of Formulas 6, 7 and 8.

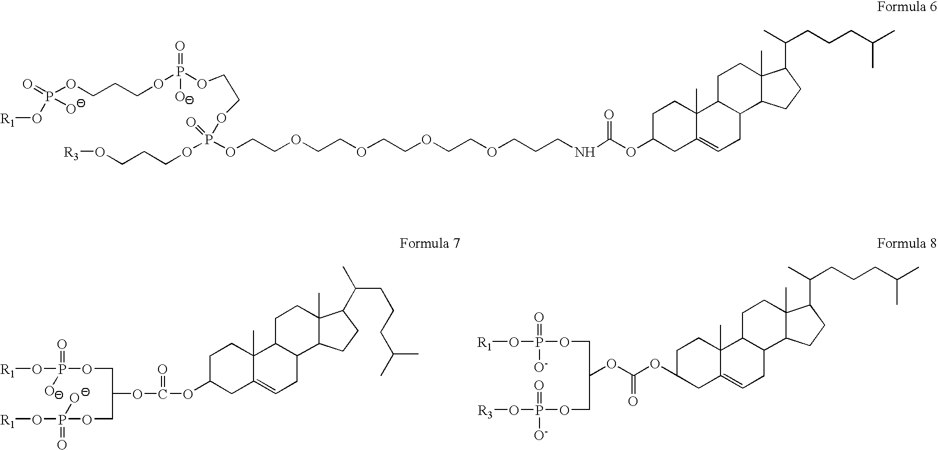

wherein R₁, R₃, R₇ and R₈ are as previously defined.

The invention further provides a chemically modified short interfering nucleic acid molecule capable of down regulating the expression of a target gene by RNA interference, wherein the molecule comprises a trivalent linker having three terminal ends, wherein a first oligonucleotide is attached to a first terminal end and a second oligonucleotide is attached to a second terminal end and a solid support matrix is attached to a third terminal end and wherein at least one of said first or second oligonucleotides, is complementary to or homologous with, the target gene. In one embodiment, the molecule of the invention has the structure of Formula 9:

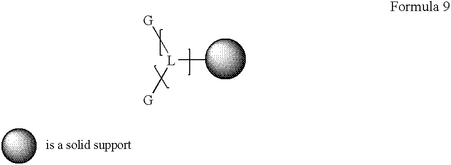

wherein G and L are previously defined.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of mediating RNA interference (“RNAi”) or gene silencing. Non-limiting examples of siRNA molecules of the invention are shown in FIGS. 2, 3, 4 and 5. For example the siRNA can be a double-stranded oligonucleotide molecule comprising a sense oligonucleotide and an antisense oligonucleotide, wherein the antisense region comprises complementarity to a target nucleic acid molecule. The siRNA can be a single-stranded hairpin oligonucleotide having self-complementary sense and antisense regions, wherein the antisense region comprises complementarity to a target nucleic acid molecule. As used herein, siRNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. In certain embodiments, short interfering nucleic acids do not require the presence of nucleotides having a 2′-hydroxy group for mediating RNAi and as such, short interfering nucleic acid molecules of the invention optionally do not contain any ribonucleotides (e.g., nucleotides having a 2′-OH group). The modified short interfering nucleic acid molecules of the invention can also be referred to as short interfering modified oligonucleotides “siMON.” As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post-transciptional gene silencing.

By “inhibit” or “down regulate” it is meant that the activity of a gene expression product or level of RNAs or equivalent RNAs encoding one or more gene products is reduced below that observed in the absence of the nucleic acid molecule of the invention. In one embodiment, inhibition with a siRNA molecule preferably is below that level observed in the presence of an inactive or attenuated molecule that is unable to mediate an RNAi response. In another embodiment, inhibition of gene expression with the siRNA molecule of the instant invention is greater in the presence of the siRNA molecule than in its absence.

By “gene” or “target gene” is meant, a nucleic acid that encodes an RNA, for example, nucleic acid sequences including, but not limited to, structural genes encoding a polypeptide. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus.

By the term “non-nucleotide” or “non-nucleotidic” is meant any group or compound which can be incorporated into a nucleic acid chain in the place of one or more nucleotide units, including either sugar and/or phosphate substitutions, and allows the remaining bases to exhibit their enzymatic activity. The group or compound may be described as abasic in that it does not contain a commonly recognized nucleotide base, such as adenosine, guanine, cytosine, uracil or thymine, and therefore lacks a base at the 1′-position. Examples of non-nucleotidic moeties are described herein and can also be found in U.S. Pub. Nos. US 2003/0206887 A1 and US 2003/0130186 A1 incorporated herein by reference.

By the term “non-nucleotidic trivalent linker” is meant any group or moiety that is not a nucleotide and comprises three points of substitution and/or attachment of additional chemical moieties. Linkers include aliphatic and aromatic moieties and can be interrupted by zero, one, two, three or more heteroatoms or functional groups for example, a trivalent linker can be a glycol moiety (or other 1, 2, 3 trioxy alkane). Preferred trivalent linkers can be manufactured form compounds which facilitate branching such as phosphates and amines.

An “aliphatic spacer” is a non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic linker is preferably straight, but may be branched or cyclic and preferably contains at least about 8 to about 24 carbon atoms, more typically between about 10 and about 20 carbon atoms. In addition to aliphatic hydrocarbons, aliphatic linkers include, polyalkoxyalkyls, such as polyalkylene glycols, polyamines, polyimines, polymethanes, polyesters and polyamide, for example. Such aliphatic groups may be further substituted. Examples of preferred aliphatic spacers include polyethylene glycol polypropoxys, polyethyleneamine, and alkyl.

An “aralkyl” spacer moiety comprises any combination of aryl groups covalently joined to alkyl groups. The alkyl groups may comprise any combination of carbon atoms, hydrogen atoms, halogen atoms, oxygen, nitrogen or other atoms, and optionally contain one or more units of unsaturation, e.g., double and/or triple bonds. The aryl groups may comprise heteroaryl groups such as those aromatic ring systems containing at least one heteroatom such as nitrogen, oxygen and sulfur. Both the alkyl groups and the aryl groups of the aralkyl spacer moiety may be further substituted. Examples of aralkyl groups include.

Hydrophobic moieties include for example, aliphatic or aromatic groups having for example, at least about 4, 6, or 8 carbons, such as butyl, pentyl, hexyl heptyl, octyl, nonyl, etc. Preferred hydrophobic moieties in accordance with the invention include cholesterol, bile acids, phospholipids, cholestanol, transferrin, or peptides comprising 1-10 amino acids or polyamine acids such as polylysine.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA, 83:9373-9377; Turner et al., 1987, J. Am. Chem. Soc., 109:3783-3785). The molecules of the invention need not be perfectly complementary (i.e. not all the contiguous residues of a nucleic acid sequence will hydrogen bond with the same number of contiguous residues in a second nucleic acid sequence). The present invention is intended to cover siRNA that comprise an antisense region or a sense region within double stranded region of the siRNA that is less than perfectly complementary to each other or to the target gene. For example, the siRNA molecules of the invention are intended to comprise bulges caused by mismatches or abasic and non-nucleotidic substitutions within the double stranded region of the siRNA. The siRNA molecules of the invention need only be as complementary as is minimally necessary to allow for RNAi catalytic activity or activity related to RNAi such as that activity associated with microRNA (miRNA) molecules (for a discussion of miRNA see, McManus et al., RNA, 6 (2002) 8420850 and Zeng et al., RNA, 9 (2003) 112-123).

The term “homology” or “homologous” as used herein, refers to the nucleotide sequence of two or more nucleic acid molecules as partially or completely identical.

By “RNA” is meant a molecule comprising at least one ribonucleotide residue. By “ribonucleotide” is meant a nucleotide with a hydroxyl group at the 2′ position of a .beta.-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or internally (e.g. capped structures), for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

The term “cap structure” as used herein, refers to chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In accordance with the invention the oligonucleotide-containing regions of the molecules of Formulas 1-5 may be further chemically modified. A chemically modified oligonucleotide region (e.g., the sense region, the antisense region, or the loop region of a hairpin molecule) may comprise modifications to the nucleotide ribose units, bases or to the oligonucleotide backbone as are known in the art; see for example US Publication Nos. US 2003/0206887 and US 2003/0130186 incorporated herein by reference. Examples of modifications to the ribose unit include the addition of one or more ribonucleotidic units where the 2′-hydroxyl group of the ribonucleotide unit is replaced by OR where R is alkyl, aryl, cycloalkyl and where one or more of the methylene units may be alternately replaced by O, NH, SO₂NH, phosphate, or sulfate groups. For example, R could be methyl in which case OR become 2′-OMe group and the ribonucleotide unit can become 2′-OMe ribonucleoside. Another example, R is CH₂CH₂OMe, in which OR becomes methoxyethoxy group.

Examples of modifications other than oligonucleotide backbone include internucleotide linkages other than phosphate such as thiosphosphate, phosphoramidate, methylphosphonate, or other than other equivalents of phosphate groups such as carboxylic, sulfonic, sulfonamido, sulfate, carbamate, urea and amide internucleotide linkages.

Other modifications include the incorporation of one on more DNA nucleotide units within the oligonucleotide. The DNA nucleotide unit may also be modified as described herein.

The oligonucleotide regions of the molecules of Formulas 1-5 may also include include the incorporation of one or more non-nucleotidic units within the oligonucleotide. Examples of suitable non-nucleotidic units include abasic molecules (e.g. sugar moieties lacking a base or having other chemical groups in place of a base at the 1′ position)

Without being bound to a particular theory, incorporation of chemical modifications in siRNA is believed to impart nuclease stability and enhance the therapeutic utility of siRNA. It is well known that incorporation of chemical modifications in antisense oligonucleotide confers it with enzymatic stability and pharmacokinetic advantages (3). Preliminary reports on chemically modified siRNA suggested that in order to maintain RNAi-competency, the siRNA molecule only tolerates certain limited modifications at the 3′-, or 5′-ends of the molecule (1) (vide infra). Interestingly, the antisense strand of the siRNA appears to be less permissive for global chemical modifications compared to the sense strand. This may be due to reduced recognition of siRNA by the proteins of the RISC thereby resulting in the lack of formation of stable RISC.

In one report, siRNA with ribonucleotide overhangs demonstrated more efficient RNAi activity compared to those with deoxy-ribonucleotide overhangs. (Hohjoh, H. RNA interference (RNAi) induction with various types of synthetic oligonucleotide duplexes, FEBS Letters, 521, 2002, 195-199). Furthermore, both sense and antisense strands are required to be ribonucleotides because the corresponding DNA/RNA hybrid duplex was devoid of RNAi activity.

It has also been reported that free 5′-OH groups on the antisense strand is required for RNA interference whereas 5′-modification of the sense strand had no effect on RNAi activity (Chiu, Y-L., Rana, T. M. RNAi in human cells: Basic Structural and Functional Features of small interfering RNA, Molecular Cell, 2002, 10, 549-561). Blocking the 3′-end had no effect on RNAi; For example, siRNA in which 3′-end of sense or antisense strand was blocked by puromycin, or biotin did not affect RNAi activity. Also siRNA with a “bulge” (extra mismatched nucleotide) in the sense strand retained RNAi activity whereas bulges in antisense strand or both strands effectively abolished RNAi activity.

The two-nucleotide overhangs in siRNA duplexes can preferably be thymidine or uridine residues. The sequence of the overhang does not appear to contribute to target recognition and RNAi activity (7).

The above findings should only be used as a general guide when designing siRNA molecules of the invention. It should be understood that the siRNA molecules of the present invention are not limited by any of these findings. Therefore, in accordance with the present invention, chemical modification of siRNA as described herein can be any modification as described herein that does not reduce or eliminate the siRNA catalytic activity. Preferred sites of chemical modification in accordance with the invention include, but are not limited to the loop region of a hairpin structure, the 5′ and 3′ ends of a hairpin structure (e.g. cap structures), the 3′ overhang regions of double stranded linear siRNA, the 5′ or 3′ ends of the sense strand and/or antisense strand of linear siRNA, and at one or more of every third nucleotide of the sense and/or antisense strand.

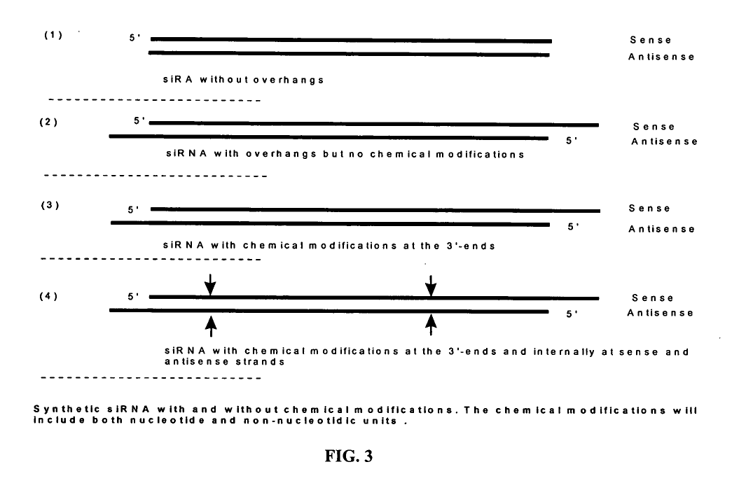

In one embodiment, the invention provides chemically modified linear siRNA molecules of Formulas 1-3. FIGS. 3 and 4 show non-limiting examples of linear siRNA in accordance with the invention. Linear siRNA in accordance with the invention comprises chemical modifications at selected sites within its sense and/or antisense strands. Preferred sites within linear siRNA include, the 3′ overhang regions of both the sense and anti sense strands of the siRNA, the 5′ and/or 3′ end of the sense and/or antisense strand, and at one or more of every third oligonucleotide of the sense and/or antisense strand. Such modifications can produce enhanced resistance to endonucleases and facilitate intracellular delivery of siRNA.

It has been reported that in the case of siRNA, efficient silencing is obtained using 21 nucleotide (NT) sense and 21 NT antisense strands paired to give 3′-overhang of two nucleotides (6,7). In general, the overhang region of siRNA is most amenable to structure modifications, and indeed the 3′-overhang can be two deoxynucleotides in both sense and antisense strands. This two nucleotide overhang makes only a small contribution to the specificity of target recognition. The overhangs are preferably symmetrical i.e., both sense and antisense strands have the same chemical composition and length. The 3′-overhang in the sense strand apparently does not contribute to recognition because it is the antisense strand that guides recognition.

It is known that the 2 to 3 NT overhangs in siRNA can tolerate structure modifications and the resulting siRNA molecule can retain RNAi competency. Potentially therefore, the length of this overhang can be increased to provide the siRNA with enhanced nuclease stability. Preferred modifications include siRNA bearing overhangs having from about 1-10 ribonucleotide units, modified ribonucleotide units, deoxyribonucleotide units, modified deoxyribonucleotide units or non-nucleotidic units. The chemically modified overhangs may be linked to the 5′ or 3′ ends of one or both sense and antisense strands via phosphate, thiophosphate, dithiophosphate, phosphoramidate, methylphosphonate, phosphoramidate or other equivalents of phosphate groups such as carboxylic, sulfonic, sulfonamido, sulfate, carbamate, urea and amide linkages. The backbone of the modified overhangs may also be modified with those same linkages as is described elsewhere herein.

One exemplary modification is the modification to the overhang region comprising a linear siRNA molecule with overhangs of 2 to 6 Thymidine, or 2 to 6 2′-OMe ribouridine units (2′-OMe U). This overhang preferably has phosphorothioate as the internucleotidic linkage. This length (2 to 6 NT) of modified nucleotide segment is known to provide substantial nuclease resistance in single-stranded oligonucleotides (17). Another preferred modification comprises an siRNA molecule with overhangs 2 to 6 non-nucleotide moieties. This length of modified non-nucleotide moieties is known to provide exonuclease resistance in single-stranded oligonucleotides and is expected to enhance intracellular delivery of siRNA. In one preferred embodiment, non-nucleotide moieties include those described by formulas 1-5 above. Non-nucleotide moieties that include ethylene glycol and cholesterol units are commercially available as phosphoramidite building blocks.

In another embodiment the invention provides chemically modified linear siRNA wherein the modifications can occur anywhere in the double stranded region of either the sense or antisense strand of the siRNA so long as the modification does not reduce the catalytic capability of the siRNA. Modifications to the siRNA include non nucleotide units such as those comprising abasic moieties, modified ribonucleotide units including 2′ deoxy modifications (DNA), or modified DNA units.

Any of the chemical modifications described above for linear siRNA molecules of the invention are also suitable for hairpin-type siRNA molecules of the invention. The hairpin siRNA structure is particularly suitable when modifications involving mismatches or bulges in the duplex region are desired. FIG. 4 shows non-limiting examples of hairpin siRNAs of the invention comprising bulges in the stem and loop regions. The hairpin structure is believed to provide enhanced stabilization of such modifications. SiRNA comprising modifications involving mismatches and bulges in the double stranded region are believed to be useful as precursor miRNA molecules in addition to being useful as siRNA molecules.

SiRNA molecules of the invention may be synthesized by standard RNA synthetic means as are known in the art and described in the examples. For further details and a discussion of the synthesis of siRNA molecules in general see, U.S Pub. No. 2003/0206887 incorporated herein by reference.

The siRNA molecules of the invention are useful for down-regulating genes associated with the onset and maintenance of a myriad of diseases for example, CNS diseases and disorders, inflammation, cardiovascular diseases, autoimmune disease, infectious disease and metabolic disorders. Thus the siRNA molecules of the invention may be adapted to prophylactically or therapeutically treat a patient with any such disease.

One exemplary embodiment is the use of the siRNA molecules of the invention to treat infectious disease such as any viral infection. In this embodiment, the antisense region of the siRNA molecule of the invention is complementary to a portion of a viral gene associated with the replication and/or pathogenesis of a viral infection. In one example of this embodiment the antisense region of the molecule is complementary to a gene from an RNA virus such as hepatitis C(HCV), HIV, West Nile Virus (WNV), Yellow Fever Virus (YFV) and Dengue virus. In another embodiment, the antisense region of the molecule is complementary to a gene encoding RdRp of an RNA virus.

In one exemplary embodiment, the siRNA molecules of the invention may be used to treat HCV infection, liver failure cirrhosis, hepatocellular carcinoma and any other indications that can respond to the level of HCV in a cell or tissue, alone or in combination with other therapies. As exemplified herein, an siRNA molecule of the invention can comprise any contiguous HCV sequence (e.g., wherein the sense region of the siNA comprises about 19 contiguous HBV nucleotides and the antisense region comprises sequence complementary to about 19 contiguous HCV nucleotides). In one embodiment, the invention features a siNA molecule having RNAi activity against HCV RNA, wherein the siNA molecule comprises a sequence complementary to any RNA having HCV encoding sequence, for example of SEQ ID NO 1 or sequences referred to in Table I and/or homologous sequences thereof. In another embodiment, the invention features a siNA molecule comprising sequences selected from the group consisting of SEQ ID NOS: 2-83.

siRNA molecules of the invention can comprise a delivery vehicle, including liposomes, for administration to a patient, carriers and diluents and their salts, and/or can be present in pharmaceutically acceptable formulations. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Beigelman et al., U.S. Pat. No. 6,395,713, and Sullivan et al., PCT WO 94/02595, further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a patient.

The invention also features pharmaceutical compositions comprising one or more siRNA molecules of the invention in a pharmaceutically acceptable carrier, such as a stabilizer, buffer, and the like. The siRNA molecules of the invention can be administered and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art. The siRNA molecules used in compositions of the invention such as those of Formulas 1-5 may include pharmaceutically acceptable forms of those molecules such as their pharmaceutically acceptable salt forms.

A pharmaceutical composition or formulation refers to a composition or formulation in a form suitable for administration, e.g., systemic administration, into a cell or patient. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

A therapeutically effective amount is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The siRNA molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more of the siRNA molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

The present invention is broadly applicable to the treatment and detection of existing and emerging viral diseases, and infectious agents in general, including those associated with bio-terrorism. As a potential therapeutic and diagnostic modality, the design and use of chemical compositions of siRNA described herein are applicable across a broad spectrum of targets and disease areas including cardiovascular, metabolic, cancer, CNS and infectious diseases. Furthermore, chemically modified siRNA described herein will be suitable for covalent attachment to solid matrices and may be ideal for designing “siRNA chips” for diagnostic applications and the study of gene function analysis directly in cultured cells.

### EXAMPLES

### Example 1

### Model Studies

As a model study, we have carried out the synthesis and preliminary evaluation of siRNA against Lamin A/C mRNA, a cytoskeletal gene transcript.

Synthesis of siRNA

As model studies against Lamin A/C target, we carried out the synthesis of modified sense and antisense strands of siRNA (given below) by automated solid-phase phosphoramidite chemistry as described in the following steps 1-2. The synthesis was done in 1 to 10 micromol scale.

5′ CUG GAC UUC CAG AAG AAC A TT (Sense)
5′ UGU UCU UCU GGA AGU CCA G TT (Antisense)
5′ CUG GAC UUC CAG AAG AAC A TpsTpsTpsT (Sense)
(chemically modified overhang)
5′ UGU UCU UCU GGA AGU CCA G TpsTpsTpsT
(Antisense) (chemically modified overhang)
5′ CUG GAC UUC CAG AAG AAC A TT cholesterol
(Sense) (chemically modified overhang with
cholesterol unit)
5′ UGU UCU UCU GGA AGU CCA G TT cholesterol
(Antisense) (chemically modified overhang with
cholesterol unit)
5′ CUG GAC UUC CAG AAG AAC A UU (Sense)
(chemically modified overhang with 2′-OMe
ribonucleoside unit)
5′ UGU UCU UCU GGA AGU CCA G UU (Antisense)
(chemically modified overhang with 2′-OMe
ribonucleoside unit)

U means 2′-OMe ribonucleoside; ps means internucleotidic phosphorothioate linkages; all other linkages are phosphoric diesters.

The appropriate building blocks are commercially available. The purified sense and antisense strands were then annealed to generate siRNA as in steps 3-5. We have prepared several unmodified and some modified siRNA using this procedure and the methodologies can be applied for the preparation of modified HCV-targeted siRNA including hairpin siRNA described in this project.

### Step 1: Assembly of RNA

The synthesis of RNA follows the same pathway as DNA synthesis and is done DMT-on or DMT-off depending on purification strategy. Most compounds can be synthesized DMT-off and then PAGE-purified. When using hydrophobic linkers, in some cases, we found it advantageous to do DMT-on synthesis, purify by reversed-phase HPLC, followed by detritylation and gel purification to get highly pure RNA.

Assembly is done as follows: A solid support with an attached building block (T or 2′-OMe U, or cholesteryl in the above case) is subjected to removal of the protecting group on the 5′-hydroxyl. The incoming amidites is coupled to the growing chain in the presence of activator. Any unreacted 5′-hydroxyl is capped, and followed up by oxidation to get phosphotriester linkage. Phosphorothioate linkage was incorporated using 3H-1,2-benzodithiole-3-one-1,1-dioxide (19). The synthesis cycle is then repeated until an oligomer of the desired length resulted.

In the case of RNA synthesis, coupling reactions between ribonucleoside phosphoramidites and support-bound nucleoside typically take 10 to 30 min for completion because of the presence of bulky 2′-OH protecting group. Reports of dramatic improvements in oligoribonucleotide synthesis have suggested the use of activators more acidic than the standard 1-H-tetrazole (pKₐ, 4.8). Such activators include 5-(4-nitrophenyl)1-H-tetrazole (pKₐ, 3.7) or 5-ethylthio-1-H-tetrazole (pKₐ, 4.28) and were used in the project when cholestyryl amidite was used.

We have carried out routine RNA synthesis up to 10 micromol scale using 2′-O-tert-butyldimethylsilyl-3′-O-(cyanoethyl-N,N-diisopropylamino)ribonucleoside phosphoramidites according to the standard procedures. Site-specific modification involved the incorporation of internucleotidic phosphorothioate linkages. We have used building blocks that carry exocyclic amino groups with regular, as well as, base-labile phenoxyacetyl or tert-butylphenoxyacetyl protecting groups.

Step 2: Deprotection and Cleavage of RNA from Solid Support

The use of base-labile protecting groups such as phenoxyacetyl, 4-isopropyl phenoxyacetyl, 4-tert-butyl phenoxy acetyl allow them to be rapidly removed after a 15 min to 1 h incubation in concentrated NH₄OH:EtOH (3:1) at 65° C. or 2-4 h at room temperature. Complete removal of standard nucleobase protecting groups could be done in 15 h under these conditions. Also, the cleavage from the solid support and removal of beta-cyanoethyl phosphate protecting groups is simultaneously accomplished.

The 2′-O-TBDMS groups were removed by using N-tetrabutyl ammonium fluoride (TBAF) (1 M in THF) at room temperature over 24 h. The use of this deprotecting agent produces salt that must be removed before analysis and purification (see step 3). The use of neat triethylamine trihydrofluoride (TEA.3HF) as desilylating reagent has also been reported. A solution of TEA.3HF in N-methylpyrrolidinone or N,N-dimethyl formamide allows full deprotection to be achieved at 65° C. or 4-8 h at room temperature and could be applied if needed.

We have used commercially available RNA monomers for RNA synthesis. Thus, following the synthesis of sense and antisense RNA according to the above protocol, each compound was purified and siRNA prepared according to Steps 3-5.

### Step 3: Purification

The crude RNA was desalted using Sephadex G-10 (NAP) column and purified further by preparative PAGE (12% acrylamide). Final desalting using NAP column followed by lyophilization gave analytically pure materials (as determined by ion-exchange HPLC) suitable for further work. The molecular weight of each sense and antisense strand was confirmed by electrospray MS, and where applicable, the presence of modified internucleotide linkages was ascertained by ³¹P-NMR (e.g., PS, □ 56 ppm)). Typically one micromol scale synthesis gave ca. 1 mg of highly pure siRNA (97% pure) following annealing of sense and antisense strands.

Ion-exchange HPLC was performed using Dionex PA-100 column. Buffer A: De-ionized water. Buffer B: 0.2 M NaOH. Buffer C: 2 M NaCl. Photodiode array detector was set at 254-260 nm. Injection volume of 20 microliter of 0.2 O.D. units was used. Linear gradient 0 to 30 min was used as follows:

| Time (min) | flow rate (ml/min) | % A | % B | % C |
|---|---|---|---|---|
| 0 | 1.2 | 82.5 | 12.5 | 5 |
| 20 | 1.2 | 42.5 | 12.5 | 45 |
| 25 | 1.0 | 82.5 | 12.5 | 5.0 |
| 30 | 1.0 | 82.5 | 12.5 | 5.0 |

Typical Rₜ of 21-mer single-stranded RNA was 22 minutes under these conditions.
Step 4: Annealing of Sense and Antisense Strands to Produce siRNA

Equal A₂₆₀ units (0.2 to 1 O.D. units) of sense and antisense were combined in 200 to 500 microliters of annealing buffer (150 mM NaCl, 10 mM NaH₂PO₄, 2 mM MgCl₂, pH 7.4). This mixture was heated at 90° C. for 2 min, kept at 4° C. overnight. The solution can be stored at −20° C. until use. The heating disrupts any higher aggregates that may have been formed upon lyophilization.

### Step 5: Analysis of the Duplexed Form

This was carried out by electrophoresis in 4% agarose gel under non-denaturing conditions. Molecular weight markers, as well as, single-stranded sense and antisense RNA were used as standards. We have observed significant mobility differences between the duplexed form and single-stranded forms.

This procedure, in addition to thermal denaturation analysis, is used to assess duplex stability and nuclease stability of chemically modified siRNA, qualitatively and quantitatively (17).

### Example 2

### Use of HCV as a Model System for Evaluating Therapeutic Utility of Synthetic siRNA

Hepatitis C virus is the major etiologic agent of parenterally transmitted non-A, non-B viral hepatitis. HCV infection occurs worldwide and HCV prevalence ranges from 0.4% (USA, UK) to 14% (Egypt). Available data indicates that about 150,000 HCV infections occur every year in the US. 10%-20% of those with chronic HCV infection will develop liver cirrhosis, and also have a high risk of developing hepatocellular carcinoma. HCV has emerged as a major etiologic agent associated with liver cirrhosis and hepatocellular carcinoma.

HCV infection has high prevalence rate among many high-risk groups. For example, amongst intravenous drug users, the prevalence is about 28% to 70% depending on the population studied. Up to 60% to 90% of hemophiliacs in Western countries are chronically infected with HCV. Infected blood appears to be the main sources of transmission of HCV.

Interferon-α (IFN-α) is the only therapeutic option available for the treatment of HCV infection. However, IFN-α therapy is only partially successful. Thus, only 70% of treated patients normalize alanine aminotransferase (ALT) levels in the serum and after discontinuation of IFN, 35% to 45% of these responders relapse. In general, only 20% to 25% of INF-treated patients have long-term responses to IFN. However, studies have showed that combination treatment with IFN plus Ribavirin results in sustained response in the majority of patients. Different genotypes of HCV respond differently to IFN therapy, genotype 1b is more resistant to IFN therapy than type 2 and 3. Based on all these observations and considerations, there is urgent need for the development of more effective antiviral therapy for HCV infection, which in all likelihood, would be administered as combination chemotherapy. RNA-dependent RNA polymerase (RdRp), the critical enzyme in HCV replication, lacks proofreading capabilities, leading to mutation and antiviral drug resistance. Hence, HCV replication dynamics indicate that potent combination therapy will be needed to suppress HCV replication and prevent the development of drug resistance. Therefore, there is an urgent need to develop novel anti-HCV drugs.

HCV belongs to the Flaviviridae family and is most closely related to the pestiviruses, which include hog cholera virus and bovine viral diarrhea virus (BVDV), Dengue, etc. Due to the lack of an efficient culture replication system for the virus early studies relied upon virus obtained from serum. The HCV genome is a single-stranded, positive-sense RNA of about 9,600 base pairs coding for a polyprotein of 3009-3030 amino-acids, which is cleaved co- and post-translationally by cellular and two viral proteinases into mature viral proteins. Part of the viral replication cycle within the liver involves the synthesis of a negative-strand RNA from which positive-strand RNA is synthesized.

During the virus-life cycle, several viral components are required for replication, which constitute potential targets for the development of anti-viral therapy, including the viral proteinases, the helicase, the RNA-dependent RNA polymerase (RdRp) and the 5′- and 3′-UTRs. Among these, the viral RdRp corresponding to NS5b gene is a validated target for drug design. However, unlike other viral polymerases, there has been not much published literature on the development of nucleoside or non-nucleoside analogs as inhibitors of HCV RdRp. Published crystal structure of HCV polymerase complexed with the short nucleotide chain suggests that the “active site cavity” for ligand entry and binding is quite narrow. Thus this shortcoming may be overcome by the development of synthetic siRNA as novel inhibitors of viral RdRp.

The antiviral activity of siRNA will be determined using an in vitro replicon assay that is known to support HCV replication. The replicon assay is a validated system for evaluating antiviral activities of potential anti-HCV compounds against virus-specific molecular targets, and is ideally suited for evaluating the RNAi-mediated antiviral activity and for understanding the mechanism of action of the synthetic siRNA.

(a) Step 1: Sequence Selection of siRNA

Using sequence information of NS5b HCV RNA (SEQ ID NO: 1), we will design synthetic siRNA molecules, 21 to 23-mer long that are complementary to selected region of the mRNA. It is important to keep this optimal length so that non-specific responses due to the double-stranded siRNA are minimal. Given a target RNA, the following set of rules have been derived by Tuschl et al., (see review 1b) regarding the selection of siRNA sequence and can be applied in the case of sequence selection for siRNA design for HCV.

Start 75 bases downstream from the start codon of mRNA sequence.
1) Locate the first AA dimer.
2) Record the next 19 nucleotides following the AA dimer.
3) Compare the 19-mer sequence to the appropriate genome database and only select that sequence which does not have significant homology to other genes.
4) Calculate the percentage of GC content of the AA-N19-21 base sequence. Ideally the G/C content is 50% but it must be less than 70% and greater than 30%.
5) This sequence and its complementary strand is the siRNA

We will use the HCV RdRp sequence EMBL HCV5NS5B (SEQ ID NO: 1) for designing siRNA. The protein sequence of RdRp is also known. The translation start site is the first ATG of the reported RNA sequence. Thus, the siRNA against HCV can be designed according to the above rules.

It is pertinent to mention that these are empirical rules for selection of region of mRNA for targeting and does not take into account the secondary structure of RNA. For different types of mRNA target, with varying secondary structures, these selection rules may or may not apply. For example, in the case of targeting HBV pregenomic RNA, the epsilon region of pgRNA has a stem loop structure that may or may not contain an ATG site or AA dimer unit. Consequently targeting pgRNA may be done without recourse to these rules by simply designing siRNA in which the antisense strand is complementary to a stem, loop, or bulge structure of the pgRNA.

Step 2: (a) Design of Chemically Modified SiRNA with Designed Secondary Structures

The purpose of incorporating secondary structure elements in synthetic siRNA is to maintain the integrity of duplexed RNA structure inside the cell, and to also provide resistance to nuclease-mediated degradation. In addition, the incorporation of certain hydrophobic non-nucleotidic units such as cholesterol, propane-dioxy, etc. might facilitate intracellular delivery of the siRNA. A hairpin loop type structure appears to be the simplest secondary structure that can be incorporated in the siRNA with specific chemical modifications. These chemical modifications are placed at selected sites like the loop region and ends of the siRNA (FIG. 2). Interestingly, unmodified intracellularly generated hairpin type siRNA via engineered plasmid has successfully demonstrated RNAi competency in vitro (14). The loop does not appear to impede the formation of the siRNA-RISC complex and RNA cleavage.

Chemically modified hairpin loop type structures can be envisaged as being joined by the 3′- and 5′-ends of the siRNA strands. The loop can be made hydrophilic by incorporating nucleotidic units such as thymidine, with a minimum of 5 to maximum of 8 units that would give a stable loop and also stabilize the duplex structure. Loop can also be made hydrophobic by using cholesterol, polyethylene glycol, and propane diol units. Indeed, the incorporation of some non-nucleotide units and nucleotide units are known to stabilize short hairpin RNA and DNA structures (18). The 3′ and 5′-ends of the stem can also be modified additionally by incorporating 2′-OMe ribonucleoside phosphorothioate units. These end modifications or capped structures (shown as dark appendages in FIG. 2) can further protect the strands against nuclease-mediated degradation (3).

One specific examples of such siRNA structures is the shown in Formula 6 wherein R1 and R3 are previously defined.

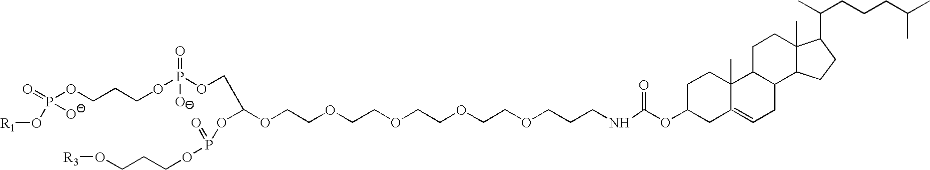

The siRNA molecule of Formula 6, siRNA with C3-cholesteryl TEG-C3-loop, may be synthesized by starting with the antisense strand 3′ to 5′ at the 5′ end add C-3 spacer (Glen 10-1913-90). Then cholesteryl triethylene glycol amidate (Glen 10-1975-95) and the spacer C-3 is added followed by the sense sequence 3′ to 5′. Compounds with additional 1 to 2 C-3 spacer units may be made using this process. In general, it should be noted that the loop can be formed by joining 3′ antisense to 5′ sense or 5′ antisense to 3′ sense as shoen in FIG. 2. The building blocks and protocols for procedures for synthesizing hairpin type siRNA of the invention are also reported (18).

Our model studies have indicated that for improved synthesis yields, in some cases, activators such as 5-(4-nitrophenyl)1-H-tetrazole (pKₐ, 3.7) or 5-ethylthio-1-H-tetrazole (pKₐ, 4.28) may be needed especially when using non-nucleoside amidites as has been reported by others (see review 11).

Step 2: (b) “Linear” siRNA with Site-Specific Chemical Modifications within the Duplex

In addition to siRNA hairpin structures with chemical modifications, this invention describes “linear” siRNA, which contain various chemical modifications at selected sites within its sense and antisense strand. Such modifications can produce enhanced resistance to endonucleases. As mentioned before, siRNA appears less permissive for global structure modifications within the double-stranded motif in maintaining RNAi-competency. We describe here certain analogs that carry one to two 2′-OMe ribonucleoside phosphorothioate and deoxyribonucleoside phosphorothioate units at selected sites within the duplex (see FIG. 3). These modifications are placed in both strands (see FIG. 3). Placement of 2′-OMe ribonucleoside phosphorothioate segment is known to enhance resistance of oligonucleotides against nuclease-mediated degradation in vitro and in vivo (13). The expedite synthesizer is convenient to do site-specific modifications where both oxidizing and sulfurizing agents can be used as needed. Care needed to be taken that adequate washings of lines are done to avoid reagent mixing and decomposition of sulfurizing reagent.

Step 2 (c) “Linear” siRNA with Overhangs

### (i) Unmodified Overhangs:

It has been reported that in the case of siRNA, efficient silencing is obtained using 21 NT sense and 21 NT antisense strands paired to give 3′-overhang of two nucleotides (6,7). In general, the overhang region of siRNA is most amenable to structure modifications, and indeed the 3′-overhang can be two deoxynucleotides in both sense and antisense strands. This two nucleotide overhang makes only a small contribution to the specificity of target recognition. The overhangs need to be symmetrical i.e., both sense and antisense strands should have the same chemical composition and length. The 3′-overhang in the sense strand apparently does not contribute to recognition because it is the antisense strand that guides recognition. An siRNA with thymidine overhangs of 2 to 6-mer in length (see FIG. 3) is synthesized. For convenience, overhang synthesis in one machine is done in one machine and the synthesis is completed in another machine.

(ii) Chemically Modified Overhangs of siRNA:

It is known that the 2 to 3 overhangs in siRNA can tolerate structure modifications and the resulting siRNA molecule can retain RNAi competency. Potentially therefore, the length of this overhang can be increased along with chemical modifications to provide it with enhanced nuclease stability. The following will be made (see FIG. 3):
(a) siRNA with overhang of length 2 to 6 T, or 2 to 6 2′-OMe ribouridine units (2′-OMe U). The overhang will have phosphorothioate as the internucleotidic linkage. This length of modified nucleotide segment is known to provide substantial nuclease resistance in single-stranded oligonucleotides (17);
(b) siRNA with overhangs of length 2 to 6 non-nucleotide moieties. This length of modified non-nucleotides is known to provide exonuclease resistance in single-stranded oligonucleotides and perhaps might assist in better intracellular delivery of sRNA. Non-nucleotide moieties include ethylene glycol and cholesterol units and these are commercially available as phosphoramidite building blocks.

It may be advantageous to use reversed-phase HPLC to get a better handle on an initial purification of the DMT-on product. This product after detritylation can then be further purified by PAGE.

FIG. 2 Design of chemically modified hairpin type siRNA. The building blocks and protocols for their incorporation are established and also reported (18).

### Example 3

### Evaluate and Select the Synthetic siRNA Compounds Based Upon to Assess Duplex Stability and Increased Nuclease Stability Compared to Unmodified or Natural RNA

### Step 1

We have developed optimal conditions for annealing of sense and antisense strand (see preliminary results). The annealed products are analyzed by thermal denaturation, and electrophoretic methods for assessment of duplex stability.

Tₘ is a useful measure of duplex stability and can be used to rank the compounds within each type of siRNA. These methods are standard (17, 18, 21) and are used to ascertain if the non-nucleotidic linkages on the siRNA with hairpin loop or siRNA with overhangs have any destabilizing influence (and the degree, if any) on the siRNA duplex. The thermal melting profiles is also used to extract thermodynamic parameters for quantitative assessment (17, 18, 21). In case of solubility problems, add small amounts of acetonitrile to the aqueous solution.

### Step 2

The synthetic siRNA compounds is evaluated for enhanced enzymatic stability by comparison with the corresponding unmodified siRNA. It is expected that conditions (such as the presence of serum components) that destabilize the duplex structure will cause strand separation and consequent exonuclease-promoted degradation of the RNA, which can then be assessed. The in vitro stability is evaluated in human serum at four time points over a 24 h period, using standard procedures (15) and duplex stability is monitored by electrophoresis. Compounds are ranked according to the stability characteristics. In the event, the stability ranking of certain duplexed siRNA by in vitro assay in serum is not possible, where needed, use the stability of the antisense strand as the criteria. The serum stability of oligonucleotides in vitro is a reasonably good predictor of stability in vivo (17). Furthermore, the plasma and tissue half-life is a key determinant of RNAi-competency of siRNA in vivo.

### Procedure for Nuclease Stability Assessment:

Typically 0.4 O.D. units of siRNA are taken up in 70 microliters of Tris-HCl buffer (25 mM, pH 7.0) to which is added 30 microliter human serum (GIBCO BRL) and incubated at 37° C. At designated time points of 1, 4 and 24 hours, aliquots are removed and treated with 2×Pk buffer. After incubation at room temperature for one hour, the reaction is extracted with phenol/chloroform/isoamyl alcohol (25:24:1). The siRNA is precipitated with 3 volumes of ethanol and analyzed by non-denaturing gel electrophoresis.

### Step 3

The compounds chosen for antiviral screening, is based on the criteria of (a) relative duplex stability (b) enhanced stability compared to unmodified RNA. SiRNA, which have issues of duplex stability or nuclease stability, is not considered for further evaluation. Both criteria along with antiviral activity is important for being considered for selection of antiviral lead(s) for further evaluation in Phase II.

### Example 4

### Screen Compounds, from Item (2) Above, in the HCV Replicon Assay for Potent, Selective, and Specific Down Regulation of HCV mRNA. This Assay also Enables Selection of those Synthetic siRNA Molecules that are Functionally RNAi-Competent

The siRNA compounds selected from above is screened initially at a single 10-micromolar doses in the HCV replicon assay in a primary assay. The replicon assay is a validated assay for evaluating inhibition of HCV replication by compounds. The evaluation is carried out using transfection reagent siPORT™ Amine (Ambion). Typical protocol for the preparation of siRNA complexed with transfection reagent has been described (7).

A summary of the assay procedure to be followed is given below:

### Primary In Vitro Cell-Based Anti-HCV Assay:

The antiviral activity of test compounds are assayed in the stably HCV RNA replicating cell line, AVA5, derived by transfection of the human hepatoblastoma cell line, Huh7 (Blight, et al., 2000, Science, 290:1972). Compounds are added to living cultures once daily for three days (media is changed with each addition of compound). Cultures generally start the assay at 50% confluence and reach confluence during the last day of treatment. HCV RNA and cellular beta-actin RNA levels are assessed, 24 hours after the last dose of compound, using dot blot hybridization. A total of 6 untreated control cultures, and triplicate cultures treated with 10 IU/mL alpha-interferon (the approximate EC₉₀ with no cytotoxicity) and 100 micromolar Ribavarin (the approximate CC₉₀ with no antiviral activity) serve as positive antiviral and toxicity controls.

Both HCV and beta-actin levels in the treated cultures are expressed as a percentage of the mean RNA levels detected in untreated cultures. beta-Actin RNA levels are used as a measure of toxicity, and to normalize the amount of cellular RNA in each sample. A level of 30% or less of HCV RNA (relative to control cultures) is considered to be a positive antiviral effect, and a level of 50% or less beta-actin RNA (relative to control cultures) is considered to be a cytotoxic effect.

Appropriate controls are added to demonstrate that observed activity is siRNA-dependent. The negative controls will include amongst others single-stranded sense, antisense, siRNA sequence with 2 to 4 mismatches and transfection reagent. In the case of modified siRNA with non-nucleotide units, the corresponding non-nucleoside units will also be included in the assay as control. We will identify the best five actives that cause 50% inhibition at 10 micromolar or lower without causing cellular toxicity.

### Example 4

### Further Evaluate Selectivity and Specificity Attributes of Active siRNA Compounds Through Dose-Response and Toxicity Assays and Grade them by Safety (CC₅₀/EC₅₀) and Selectivity Indices

This study is carried out in a secondary in vitro anti-HCV assay. Dividing cultures of AVA5 cells are treated once daily for three days (media is changed with each addition of compound) with four concentrations of test compound (three cultures per concentration). A total of six untreated control cultures, and triplicate cultures treated with 10, 3, and 1 IU/ml alpha-interferon (active antiviral with no cytotoxicity) and 100, 10, and 1 micromolar ribavarin (no antiviral activity and cytotoxic) serve as controls. HCV RNA and cellular beta-actin serve as controls. HCV RNA and cellular beta-actin RNA levels are assessed 24 hours after the last dose of compound using dot blot hybridization. Beta actin RNA levels are used to normalize the amount of cellular RNA in each sample.

Toxicity analyses are performed on separate plates from those used for the antiviral assays. Cells for cytotoxicity analyses are cultured and treated with test compounds with the same schedule and under identical culture conditions as used for antiviral evaluations. Each compound is tested at four concentrations, each in triplicate cultures. Uptake of neutral dye is used to determine the relative level of toxicity 24 hours following the last treatment. The absorbance of internalized dye at 510 nM (A₅₁₀) is used for quantitative analysis. Values in test cultures are compared to nine cultures of untreated cells maintained on the same plate as the test cultures.

The 50% and 90% effective antiviral concentrations (EC₅₀, EC₉₀) and the 50% cytotoxic concentrations (CC₅₀) are calculated and used to generate Selectivity Indexes (CC₅₀/EC₅₀). A selectivity index of 10 or greater for a compound is considered for selective antiviral effect. The lead compound(s) is selected based on this criteria.

It is likely that the herein described synthetic siRNA can be delivered to virus-infected cells without the aid of transfection reagent. Therefore, the antiviral activity of active siRNA compounds from the above assay is also evaluated without the aid of delivery agent. This is particularly useful to see if chemically modified siRNA (e.g. that with non-nucleotide units) is amenable to cell-culture studies without the aid of delivery reagents.

### LITERATURE CITED

1) For reviews of RNA interference, see: (a) Hannon, G. J., “RNA interference,” Nature, 2002, 418, 244-251. (b) Tuschl, T., “RNA interference and small interfering RNAs,” CHEMBIOCHEM., 2001, 2, 239-245. (c) Zamore, P. D., “Ancient pathways programmed by small RNAs,” Science, 2002, 296, 1265-1269. (d) Sharp, P. A., “RNA interference,” Genes Dev., 2001, 15, 485-490.
2) Blight et al., Science, 1972, 29, 200.
3) For reviews on antisense approach see: (a) Mirabelli, C. T., Crooke, S. T. “Antisense Oligonucleotides in the context of modern molecular drug discovery and development,” In: Antisense Research and Applications, S. T. Crooke and B. Lebleu (Eds.) CRC Press, New York 1993, 7-35. (b) Szymkowski, D. E. “Developing antisense oligonucleotides from the laboratory to clinical trials,” Drug. Disc. Today, 1996, 1, 415-28 (c) Agrawal, S., Iyer, R. P., “Perspectives in antisense therapeutics,” Pharmacol. Therap., 1997, 76, 151-60.
4) Jorgensen, R., “Altered gene expression in plants due to trans interactions between homologous genes,” Trends Biotechnol., 1990, 8, 340-344.
5) Fire et al., “Potent and specific genetic interference by double-stranded RNA in caenorhabditis elegans,” Nature, 1998, 391, 806-811.
6) Tuschl, T, Zamore P. D., Lehmann, R., Bartel D. P, Sharp P. A., “Targeted mRNA degradation by double-stranded RNA in vitro,” Genes Dev., 1999, 13, 3191-319.
7) Elbashir S. M., Harborth, J., Lendeckel, W. Yalcin, A. Weber, K. Tuschl, T., “Duplexes of 21-nucleotide RNAs mediate RNA interference in cultured mammalian cells,” Nature, 2001, 411, 494-498.
8) Zamore P. D., Tuschl, T. Sharp, P. A., Bartel D. P., “RNAi: double-stranded RNA directs the ATP-dependent cleavage of mRNA at 21 to 23 nucleotide intervals,” Cell, 2000, 101, 25-33.
9) Usman, N., Ogilvie, K. K., Jiang, M. Y., Cedergren, R. J., J. Am. Chem. Soc., 1987, 109, 7845-7854.
10) Beaucage, S. L., Caruthers, M. H., Tetrahedron Lett., 1981, 22, 1859-1862. For a review see: Beaucage, S. L., Iyer, R. P., Advances in the synthesis of oligonucleotides by the phosphoramidite approach, Tetrahedron, 1992, 48, 2223-2311.
11) For reviews, see: Beaucage, S. L., Iyer, R. P., (a) The synthesis of specific ribonucleotides and unrelated phosphorylated biomolecules by the phosphoramidite method. Tetrahedron, 1993, 49, 10441-10488 (b) Beaucage, S. L., Iyer, R. P. “The synthesis of modified oligonucleotides by the phosphoramidite approach and their applications,” Tetrahedron, 1993, 49, 6123-6194 (c) Beaucage, S. L., Iyer, R. P., “The functionalization of oligonucleotides via phosphoramidite derivatives,” Tetrahedron, 1993, 49, 1925-1963.
12) Scaringe, S. A., Wincot, F. E., Caruthers, M. H., “Novel RNA synthesis method using 5′-O-silyl 2′-orthoester protecting groups,” J. Am. Chem. Soc. 1998, 120, 11820-21.
13) Zhang, R., Lu, Z., Zhao, H., Zhang, X., Diasio, R. B., Habus, I., Jiang, Z., Iyer, R. P., Yu, D., Agrawal, S., “In vivo stability and metabolism of a “hybrid” oligonucleotide phosphorothioate in rats,” Biochem Pharmacol., 1995, 50, 545-56.
14) (a) Brummelkamp, T. J., Bernards, R., and Agami, R., “A system for stable expression of short interfering RNAs in mammalian cells,” Science, 2002, 296, 550-553; (b) McManus, M. T., Petersen, C. P., Haines, B. B., Chen, J., Sharp, P. A., “Gene silencing using micro-RNA designed hairpins,” RNA, 2002, 1, 842-850.
15) Iyer, R. P., Yu, D., Agrawal, S. Prodrugs of Oligonucleotides. The acyloxyalkyl esters of oligodeoxyribonucleoside phosphorothioates. Bioorg. Chem. 1995, 23, 1-21.
16) Byrom, M., Pallotta, V., Brown, D., Ford, L., “Visualizing siRNA in mammalian cells: Fluorescence Analysis of the RNAi effect,” Ambion Technotes Newsletter, 2002, 9, No. 3.
17) Yu, D., Iyer, R. P., Shaw, D. R., Lisziewicz, J., Li, Y., Jiang, Z. Roskey, A., Agrawal, S., “Hybrid Oligonucleotides: synthesis, biophysical properties, stability studies, and biological activity,” Bioorganic Medicinal Chemistry, 1996, 4, 1685-92.
18) Nelson, J. S., Ellington, A. D., Letsinger, R. L., “Incorporation of a non-nucleotide bridge into hairpin oligonucleotides capable of high-affinity binding to the Rev protein of HIV-1,” Biochemistry, 1996, 35, 5339-5344.
19) Iyer, R. P., Egan, W., Regan, J. B., Beaucage, S. L., “3H-1,2-benzodithiole-3-one-1,1-dioxide as an improved sulfurizing reagent in the solid-phase synthesis of oligodeoxynucleoside phosphorothioates,” J. Am. Chem. Soc., 1990, 112, 1253-54.
20) Craggs, J. A., Ball, J. K., Thomson, B. J., Irving, W. L., Grabowska, A. M., “Development of a strand-specific RT-PCR based assay to detect the replicative form of hepatitis C virus RNA,” J. Viro. Methods, 2001, 94 (1-2), 111-120.
21) Plum, E. G., “Biophysical analysis of nucleic acids: Optical method,” In: Current Protocols in Nucleic Acids Chemistry, Beaucage, Bergstrom, Glick, Jones (Eds.) John Wiley & Sons. New York.

The patent and scientific literature referred to herein establishes the knowledge that is available to those with skill in the art. The issued U.S. patents, allowed applications, published foreign applications, and references herein are hereby incorporated by reference.

While this invention has been particularly shown and described with references to preferred embodiments thereof, it will be understood by those skilled in the art that various changes in the form and details may be made therein without departing form the scope of the invention encompassed by the appended claims.

TABLE 1
HCV siRNA Sequences (antisense regions)

| | |
|---|---|
| siRNA Antisense Sequence/start | |
| position (bp#)/GC content (%) | SEQ ID NO |

| | | | |
|---|---|---|---|
| aagccagctcgccttatcgtatt | 463 | 48% | 2 |
| aaggtcgctcacagagcggcttt | 759 | 57% | 3 |
| aaggatgatcctgatgactcatt | 1263 | 39% | 4 |
| aagggtgtactatctcacccgtg | 1137 | 52% | 5 |
| aaatggccctttacgatgtggtc | 515 | 48% | 6 |
| aatgacatccgtgttgaggagtc | 691 | 48% | 7 |
| aaggaccaagctcaaactcactc | 1590 | 48% | 8 |
| aaccagaatacgacttggagttg | 1064 | 43% | 9 |
| aaggagatgaaggcgaaggcgtc | 205 | 57% | 10 |
| aaggccgttaaccacatccgctc | 340 | 57% | 11 |
| aaaaaatgccctatgggcttctc | 631 | 43% | 12 |
| aaaggggcagaactgcggctatc | 807 | 57% | 13 |
| aaatgccctatgggcttctcata | 634 | 43% | 14 |
| aacatggtctatgctacaacatc | 1033 | 9% | 15 |
| aaaaagccctagattgtcagatc | 1319 | 39% | 16 |
| aaagccctagattgtcagatcta | 1321 | 39% | 17 |
| aatacctggaaatcgaaaaaatg | 616 | 30% | 18 |
| catgtggtgcctactcctacttt | 1716 | 48% | 19 |
| gactccatggccttagcgcattt | 1394 | 52% | 20 |
| catcaatgcactgagcaactctt | 66 | 43% | 21 |
| caaccagaatacgacttggagtt | 1063 | 43% | 22 |
| cacttgacctacctcagatcatt | 1367 | 43% | 23 |
| cagttggatttatccagctggtt | 1630 | 43% | 24 |
| taaggtcgctcacagagcggctt | 758 | 57% | 25 |
| catcgggggccccctgactaatt | 783 | 61% | 26 |
| tagcgcattttcactccatagtt | 1407 | 39% | 27 |
| cacattcggccaaatctaaattt | 281 | 35% | 28 |
| catcatggcaaaaaatgaggttt | 411 | 35% | 29 |
| gacgcggcgagcctacgagcctt | 994 | 70% | 30 |
| caccaattgacaccaccatcatg | 395 | 48% | 31 |
| cactgagaatgacatccgtgttg | 684 | 48% | 32 |
| cacatgttacttgaaggcctctg | 879 | 48% | 33 |
| catatatcacagcctgtctcgtg | 1677 | 48% | 34 |
| caacggtcactgagaatgacatc | 677 | 48% | 35 |
| catgctcctccaatgtgtctgtc | 1094 | 52% | 36 |
| cattgagccacttgacctacctc | 1359 | 52% | 37 |
| cagtaaggaccaagctcaaactc | 1586 | 48% | 38 |
| cattcaacgactccatggcctta | 1386 | 48% | 39 |
| catgcctcaggaaacttggggta | 1460 | 52% | 40 |
| gaaggacttgctggaagacactg | 369 | 52% | 41 |
| gagatcaatagggtggcttcatg | 1441 | 48% | 42 |
| gaaggcgtccacagttaaggcta | 219 | 52% | 43 |
| gaacctatccagcaaggccgtta | 327 | 52% | 44 |
| gatgcatctggcaaaagggtgta | 1123 | 48% | 45 |
| tatgacacccgctgctttgactc | 655 | 52% | 46 |
| tactttctgtaggggtaggcatc | 1733 | 48% | 47 |
| caccaccatcatggcaaaaaatg | 405 | 43% | 48 |
| caatgtgtctgtcgcgcacgatg | 1104 | 57% | 49 |
| caaactcactccaatcccggctg | 1602 | 57% | 50 |
| catggtctatgctacaacatctc | 105 | 43% | 51 |
| cagatctacggggcctgttactc | 1336 | 57% | 52 |
| caggccgtgatgggctcttcata | 550 | 57% | 53 |
| gaggagtcaatctaccaatgttg | 706 | 43% | 54 |
| gataacatcatgctcctccaatg | 1086 | 43% | 55 |
| gattgtcagatctacggggcctg | 1330 | 57% | 56 |
| gaagccagacaggccataaggtc | 742 | 57% | 57 |
| gagttgataacatcatgctcctc | 1081 | 43% | 58 |
| gactcatttcttctccatccttc | 1278 | 43% | 59 |
| gaaggcgaaggcgtccacagtta | 213 | 57% | 60 |
| tacgagccttcacggaggctatg | 1007 | 57% | 61 |
| taacatcatgctcctccaatgtg | 1088 | 43% | 62 |
| tacctcagatcattcaacgactc | 1376 | 43% | 63 |
| taggggtaggcatctacctgctc | 1742 | 57% | 64 |
| tactcctactttctgtaggggta | 1727 | 43% | 65 |
| caacatctcgcagcgcaagcctg | 119 | 61% | 66 |
| caatctaccaatgttgtgacttg | 713 | 39% | 67 |
| caggactgcacgatgctcgtgtg | 925 | 61% | 68 |
| caacttgaaaaagccctagattg | 1312 | 39% | 69 |
| caagcctgcggcagaagaaggtc | 134 | 61% | 70 |
| caaaaaatgaggttttctgcgtc | 419 | 39% | 71 |
| cagaatacgacttggagttgata | 1067 | 39% | 72 |
| gactagatactctgccccccctg | 1029 | 61% | 73 |
| gacttggagttgataacatcatg | 1075 | 39% | 74 |
| gaagtgtccgcgctaggctactg | 1514 | 61% | 75 |
| tacttgaaggcctctgcggcctg | 886 | 61% | 76 |
| tacgacttggagttgataacatc | 1072 | 39% | 77 |
| cagaactgcggctatcgccggtg | 814 | 65% | 78 |
| caatcccggctgcgtcccagttg | 1613 | 65% | 79 |
| gacagcgggtcgagttcctggtg | 593 | 65% | 80 |
| cagggggggagggctgccacttg | 1540 | 74% | 81 |
| gaaggggggccgcaagccagctc | 450 | 74% | 82 |
| caccaccccccttgcgcgggctg | 1164 | 78% | 83 |

What is claimed is:

1. A chemically modified short interfering nucleic acid molecule capable of down regulating the expression of a target gene by RNA interference, wherein said molecule comprises a non-nucleotidic trivalent linker having three terminal ends, wherein a first oligonucleotide is attached to a first terminal end and a second oligonucleotide is attached to a second terminal end and a hydrophobic moiety is attached to a third terminal end and wherein at least one of said first or second oligonucleotides, is complementary to or homologous with, the target gene.

2. The molecule of claim 1, wherein said hydrophobic moiety is attached to a terminal end of the trivalent linker via a spacer moiety.

3. The molecule of claim 1, wherein said spacer moiety is a long-chain spacer moiety.

4. The molecule of claim 3, wherein said spacer moiety is an aliphatic moiety or substituted aliphatic moiety optionally interrupted by one or more heteroatoms.

5. The molecule of claim 3, wherein said spacer moiety is an aralkyl moiety or substituted aralkyl moiety optionally interrupted by one or more heteroatoms.

6. The molecule of claim 1, wherein said trivalent linker is a glycol moiety, an alkanol amine moiety, a phosphate moiety, a sulfonamide moiety, or a carbamate moiety.

7. The molecule of claim 1, wherein said first oligonucleotide attached to the first terminal end is an antisense oligonucleotide that is complementary to a target gene and to the second oligonucleotide attached to the second terminal end.

8. The molecule of claim 1, wherein the first oligonucleotide attached to the first terminal end is a sense oligonucleotide that is complementary to the second oligonucleotide attached to the second terminal end.

9. The molecule of claim 1 further comprising a third oligonucleotide wherein the first and second oligonucleotides, when taken together with the trivalent linker to which they are attached, are complementary to a portion the third oligonucleotide and form a double stranded siRNA with the third oligonucleotide.

10. A chemically modified short interfering nucleic acid molecule capable of down regulating the expression of a target gene by RNA interference having the structure of Formula 1: 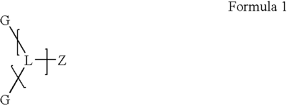
wherein each G is independently an oligonucleotide;
L is a trivalent linker; and
Z is a hydrophobic moiety; and wherein at least one G is complementary to or homologous with a target gene.

11. The molecule of claim 10, wherein said hydrophobic moiety is attached to a terminal end of the trivalent linker via a spacer moiety.

12. The molecule of claim 11, wherein said spacer moiety is a long-chain spacer moiety.

13. The molecule of claim 12, wherein said spacer moiety is an aliphatic moiety or substituted aliphatic moiety optionally interrupted by one or more heteroatoms.

14. The molecule of claim 12, wherein said spacer moiety is an aralkyl moiety or substituted aralkyl moiety optionally interrupted by one or more heteroatoms.

15. The molecule of claim 10, wherein said trivalent linker is a glycol moiety, an alkanol amine moiety, a phosphate moiety, a sulfonamide moiety, or a carbamate moiety.

16. A chemically modified short interfering nucleic acid molecule capable of down regulating the expression of a target gene by RNA interference, wherein the molecule comprises a single-strand hairpin structure having a loop region and having self-complementary sense and antisense oligonucleotide regions wherein the antisense region is complementary to a portion of the target gene and wherein the loop region comprises a non-nucleotidic trivalent linker substituted with a hydrophobic moiety.

17. A chemically modified short interfering nucleic acid molecule capable of down regulating the expression of a target gene by RNA interference, wherein the molecule comprises the structure of Formula 2: 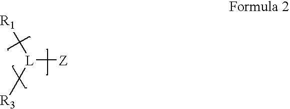
wherein R1 is an antisense or sense oligonucleotide complementary to R3, wherein when R1 is an antisense oligonucleotide, R1 is also complementary to the target gene;
R3 is a sense or antisense oligonucleotide complementary to R1, wherein when R3 is an antisense oligonucleotide, R3 is also complementary to a target gene;
L is a trivalent linker; and
Z is a hydrophobic moiety.

18. The molecule of claim 17, wherein one or both of the sense region and the antisense region comprise one or more chemical modifications.

19. The molecule of claim 18, wherein either or both of the sense region and the antisense region comprise a chemical modification at one or more of every third nucleotide beginning at the 5′ end of the oligonucleotide.

20. The molecule of claim 18, wherein the chemical modifications comprise nucleic acid backbone modifications, nucleic acid sugar modifications, or nucleic acid base modifications.

21. The molecule of claim 18, wherein the chemical modification creates a bulge or mismatch in the self complementary sense or antisense region.

22. The molecule of claim 21, wherein the chemical modification is to the antisense oligonucleotide.

23. A chemically modified short interfering nucleic acid molecule capable of down regulating the expression of a target gene by RNA interference, wherein the molecule comprises the structure of Formula 4: 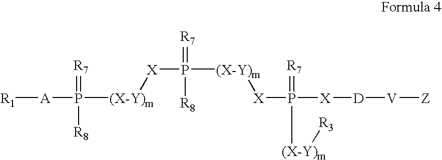
wherein R₁ is a sense or antisense oligonucleotide that is complementary to R₃ and when R₁ is an antisense oligonucleotide, R₁ is also complementary to a target gene;
R₃ is a sense or antisense oligonucleotide that is complementary to R₁ and when R₃ is an antisense oligonucleotide, R₃ is also complementary to a target gene;
A is S or O
R₇ are each independently S or O;
R₈ are each independently OH, SH, or N4R₅;
R₄, R₅, and R₆ are each independently H, alkyl, substituted alkyl, alkaryl or substituted alkaryl, aralkyl or substituted aralkyl;
X are each independently S, O or NR₄;
Y is (CH₂).
m is 0-20;
n is 1-20
D is a long-chain aliphatic linker which is optionally interrupted by one or more heteroatoms;
V is an ester moiety or an amide moiety; and
Z is a hydrophobic moiety.

24. A chemically modified short interfering nucleic acid molecule capable of down regulating the expression of a target gene by RNA interference, wherein the molecule comprises the structure of Formula 3: 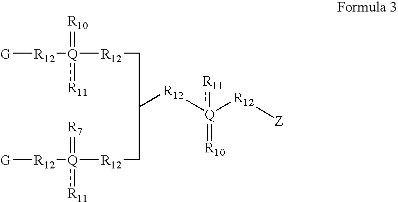
wherein each Q is independently a hexavalent atom such as sulfur, a pentavalent atom such as phosphorus, or a tetravalent atom such as sulfur, or carbon in which case the bond between Q and R11 does not exist;
each R₁₀ is optionally O, S or NH except that when Q=S, R₁₀=O, R₁₁=O and R₁₂ is optionally O or NH;
each R₁₂ is optionally O, S, or NH;
each R₁₁ is O;
G is an oligonucleotide; and
Z is a hydrophobic moiety.

25. A method of inhibiting the expression of a target gene comprising contacting cells that comprise the target gene with a chemically modified short interfering nucleic acid molecule that mediates the inhibition of the expression of a target gene, wherein said molecule comprises a non-nucleotidic trivalent linker having three terminal ends, wherein a first oligonucleotide is attached to a first terminal end and a second oligonucleotide is attached to a second terminal end and a hydrophobic moiety is attached to a third terminal end and wherein at least one of said first or second oligonucleotides, is complementary to, or homologous with, the target gene.

26. A method of inhibiting the expression of a target gene, comprising contacting cells that comprise the target gene with a molecule comprising a single-stranded hairpin structure having a loop region and having self-complementary sense and antisense polynucleotide regions wherein said antisense region is complementary to a portion of the target gene and wherein said loop region comprises a non-nucleotidic trivalent spacer substituted with a hydrophobic chemical moiety.

27. The method of claim 25 wherein said molecule has the structure of Formula 1: 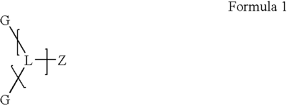
wherein each G is independently an oligonucleotide;
L is a trivalent linker; and
Z is a hydrophobic moiety; and wherein at least one G is complementary to or homologous with a target gene.

28. The method of claim 26, wherein the molecule has the structure of Formula 2: 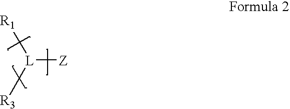
wherein R₁ is an antisense or sense oligonucleotide complementary to R₃, wherein when R₁ is an antisense oligonucleotide, R₁ is also complementary to the target gene;
R₃ is a sense or antisense oligonucleotide complementary to R1, wherein when R₃ is an antisense oligonucleotide, R₃ is also complementary to a target gene;
L is a trivalent linker; and
Z is a hydrophobic moiety.

29. The method of claim 25, wherein the molecule has the structure of Formula 3: 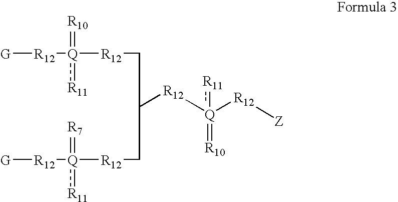
wherein each Q is independently a hexavalent atom such as sulfur, a pentavalent atom such as phosphorus, or a tetravalent atom such as sulfur, or carbon in which case the bond between Q and R₁₁ does not exist;
each R₁₀ is optionally O, S or NH except that when Q=S, R₁₀=O, R₁₁=O and R₁₂ is optionally O or NH;
each R₁₂ is optionally O, S, or NH;
each R₁₁ is O;
G is an oligonucleotide; and
Z is a hydrophobic moiety.

30. The method of claim 26, wherein the molecule has the structure of Formula 6: 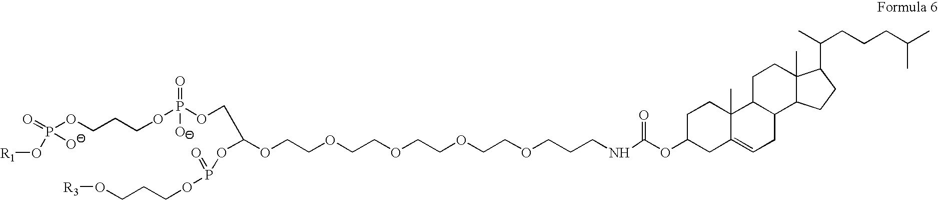
wherein R₁ is an antisense or sense oligonucleotide complementary to R₃, wherein when R₁ is an antisense oligonucleotide, R₁ is also complementary to the target gene; and
R₃ is a sense or antisense oligonucleotide complementary to R₁, wherein when R₃ is an antisense oligonucleotide, R₃ is also complementary to a target gene.

31. The method of claim 25, wherein the molecule has the structure of Formula 7: 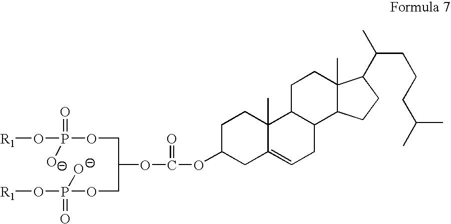
wherein R₁ is an antisense or sense oligonucleotide complementary to R₃, wherein when R₁ is an antisense oligonucleotide, R₁ is also complementary to the target gene; and
R₃ is a sense or antisense oligonucleotide complementary to R₁, wherein when R₃ is an antisense oligonucleotide, R₃ is also complementary to a target gene.

32. The method of claim 25, wherein said target gene is a gene associated with the onset or maintenance of a disease selected from the group consisting of inflammation, autoimmune disease, CNS diseases and disorders, cancer, infectious diseases and metabolic disorders.

33. The method of claim 25, wherein said target gene is a viral gene associated with replication and/or pathogenesis of a virus.

34. A pharmaceutical composition comprising a pharmaceutically acceptable excipient and a chemically modified short interfering nucleic acid molecule that down regulates expression of a target gene, wherein said molecule comprises a non-nucleotidic trivalent linker having three terminal ends, wherein a first oligonucleotide is attached to a first terminal end and a second oligonucleotide is attached to a second terminal end and a hydrophobic moiety is attached to a third terminal end and wherein at least one of said first or second oligonucleotides, is complementary to or homologous with, the target gene.

35. A pharmaceutical composition comprising a pharmaceutically acceptable excipient and a molecule comprising a single-stranded hairpin structure having a loop region and having self-complementary sense and antisense polynucleotide regions wherein said antisense region is complementary to a portion of the target gene and wherein said loop region comprises a non-nucleotidic trivalent spacer substituted with a hydrophobic chemical moiety.

36. The composition of claim 34 wherein the molecule has the structure of Formula 1. 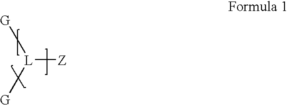
wherein each G is independently an oligonucleotide;
L is a trivalent linker; and
Z is a hydrophobic moiety; and wherein at least one G is complementary to or homologous with a target gene.

37. The composition of claim 35, wherein the molecule has the structure of Formula 2: 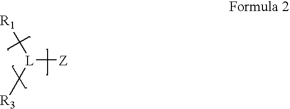
wherein R₁ is an antisense or sense oligonucleotide complementary to R₃, wherein when R₁ is an antisense oligonucleotide, R₁ is also complementary to the target gene;
R₃ is a sense or antisense oligonucleotide complementary to R1, wherein when R₃ is an antisense oligonucleotide, R₃ is also complementary to a target gene;
L is a trivalent linker; and
Z is a hydrophobic moiety.

38. The composition of claim 34, wherein the molecule has the structure of Formula 3: 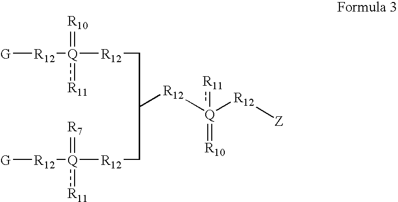
wherein each Q is independently a hexavalent atom such as sulfur, a pentavalent atom such as phosphorus, or a tetravalent atom such as sulfur, or carbon in which case the bond between Q and R₁₁ does not exist;
each R₁₀ is optionally O, S or NH except that when Q=S, R₁₀=O, R₁₁=O and R₁₂ is optionally O or NH;
each R₁₂ is optionally O, S, or NH;
each R₁₁ is O;
G is an oligonucleotide; and
Z is a hydrophobic moiety.

39. The composition of claim 1, wherein said target gene is a gene associated with the onset or maintenance of a disease selected from the group consisting of inflammation, autoimmune disease, CNS diseases and disorders, cancer, infectious diseases and metabolic disorders.

40. The composition of claim 1, wherein the target gene is a viral gene associated with replication and/or pathogenesis of a virus.

41. The composition of claim 34, wherein said molecule further comprises a third oligonucleotide, wherein the first and second oligonucleotides, when taken together with the trivalent linker to which they are attached, are complementary to a portion of the third oligonucleotide and form a double stranded siRNA with the third oligonucleotide.

42. The method of claim 25, wherein said molecule further comprises a third oligonucleotide, wherein the first and second oligonucleotides, when taken together with the trivalent linker to which they are attached, are complementary to a portion of the third oligonucleotide and form a double stranded siRNA with the third oligonucleotide.

43. A chemically modified short interfering nucleic acid molecule capable of down regulating the expression of a target gene by RNA interference, wherein said molecule comprises a trivalent linker having three terminal ends, wherein a first oligonucleotide is attached to a first terminal end and a second oligonucleotide is attached to a second terminal end and a solid support matrix is attached to a third terminal end and wherein at least one of said first or second oligonucleotides, is complementary to or homologous with, the target gene.

44. The composition of claim 35, wherein the molecule has the structure of Formula 8: 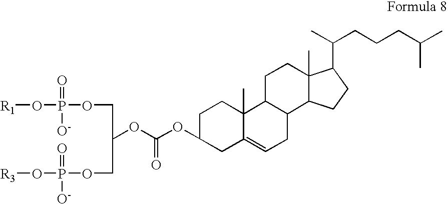
wherein R₁ is an antisense or sense oligonucleotide complementary to R₃, wherein when R₁ is an antisense oligonucleotide, R₁ is also complementary to the target gene;
R₃ is a sense or antisense oligonucleotide complementary to R₁, wherein when R₃ is an antisense oligonucleotide, R₃ is also complementary to a target gene.
